SEQUENCE LISTING (2 sequences)

SEQ ID NO: 1 - RNA, 29 nt, Staphylococcus aureus
gcauccuggggcuggagcaggucccaagg

SEQ ID NO: 2 - RNA, 29 nt, Mycobacterium tuberculosis
gcauccuggggcuguagucggucccaagg

(19) United States
(12) Patent Application Publication
(10) Pub. No.: US 2022/0055997 A1
(43) Pub. Date: Feb. 24, 2022

(54) ANTIMICROBIAL COMPOUNDS AND COMPOSITIONS COMPRISING SAME

(71) Applicant: B.G. NEGEV TECHNOLOGIES AND APPLICATIONS LTD., AT BEN-GURION UNIVERSITY, Beer Sheva (IL)

(72) Inventor: Barak AKABAYOV, Rehovot (IL)

(21) Appl. No.: 17/421,247

(22) PCT Filed: Jan. 8, 2020
(86) PCT No.: PCT/IL2020/050032

§ 371 Date: Jul. 7, 2021

Related U.S. Application Data
(60) Provisional application No. 62/822,919, filed on Mar. 24, 2019.
(60) Provisional application No. 62/789,570, filed on Jan. 8, 2019.

(51) Int. Cl. C07D 277/28; A61P 31/04

(52) U.S. Cl. CPC C07D 277/28; A61P 31/04

(57) ABSTRACT

The present invention is directed to antibacterial compositions and methods of use thereof, such as in the treatment of infectious diseases. Additionally, provided herein a method of screening molecules for identifying potential biologically active compounds.

### CROSS REFERENCE TO RELATED APPLICATIONS

This application claims the benefit of priority of U.S. Provisional Patent Application Nos. 62/789,570 filed Jan. 8, 2019 and 62/822,919 filed Mar. 24, 2019, both entitled “ANTIMICROBIAL COMPOUNDS AND COMPOSITIONS COMPRISING SAME”, the contents of which are all incorporated herein by reference in their entirety.

### FIELD OF INVENTION

The present invention is in the field of microbiology.

### BACKGROUND OF THE INVENTION

Mycobacterium tuberculosis (Mtb), among other pathogens, has been known to develop drug resistance. There is thus a pressing need to develop new and more effective antimicrobials against a range of such drug-resistant pathogens.

A good drug target is considered one that is: (a) an essential gene product; (b) structurally different from mammalian proteins (reducing mechanism-based toxicity); (c) has a reasonable site to which small molecules can bind and exert a biological effect; and (d) has a low propensity to promote rapid resistance development. In some cases, it is highly beneficiary if the target is conserved across bacterial species, and therefore the developed drug may provide a broad antibacterial spectrum. These criteria are largely met by the bactericidal target site—the ribosomal peptidyl transferase center (PTC), which resides at the heart of the gene translation machinery.

The bacterial ribosome fulfills most of the requirements of a potential drug target. Most importantly, it differs significantly from the human ribosome, and therefore constitutes a highly selective target. Further, the bacterial ribosome basic components are essential for the viability of the bacterial cell.

The PTC is a universally conserved ribonucleotide chain (rRNA) within the large ribosomal subunit (50S) that catalyzes the formation of a peptide bond during the synthesis of a peptide chain. The PTC is located in the center of the ribosome, where it forms a ‘pocket’ structure.

### SUMMARY OF THE INVENTION

The present invention, provides antibacterial compounds, compositions comprising same and methods of inhibiting or reducing bacterial growth using the disclosed compounds.

The invention is based, in part, on the findings of a family of compounds specifically targeting the bacterial peptidyl transferase center (PTC). The invention is further based, in part, on the findings that the disclosed compounds showed antibacterial activity (e.g., inhibition of protein synthesis by bacterial ribosomes, and inhibition of bacterial cell growth) equivalent to, or superior to chloramphenicol, a widely used antibiotic drug.

According to one aspect, there is provided an antibacterial composition comprising a compound or a salt thereof and an acceptable carrier being represented by the structure of general Formula A:

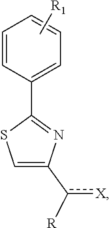

wherein R is selected from the group consisting of:

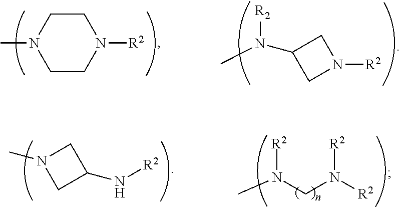

each R² independently comprises: a hydrogen atom, a linear or branched, and unsubstituted or substituted C₁-C₁₂ alkyl, aralkyl, aryl, carboxy group, imine, heteroaryl group, benzyl, a heterocyclyl, an alkyloxycarbonyl, an alkoxy group or any combination thereof, X comprises an atom selected from the group consisting of: hydrogen, carbon, oxygen, nitrogen, and sulfur or any combination thereof; [symbol] represents a double bond or a single bond; n represents an integer being in a range between 1 and 40; R¹ represents a substituent selected from the group consisting of: a halogen atom, hydrogen, a nitro group, an amine group, an azide group, a cyano group, a methoxy group, a carboxylic group, an ester group, an ether group, an aromatic group, alkyl group, vinyl group, an alcohol, a carbamate group, a thiazole group, an urea group, and an alkyl group, or any combination thereof, and wherein the compound or the salt thereof has a half maximal inhibitory concentration (IC₅₀) ranging from 1-1,200 μM.

In one embodiment, the compound is represented by general Formula I:

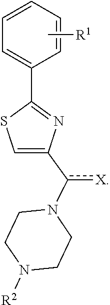

In one embodiment, X comprises an atom selected from the group consisting of: hydrogen, carbon, and oxygen, or any combination thereof.

In one embodiment, the compound is represented by general Formula Ia:

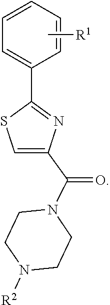

In one embodiment, the compound comprises at least one of:

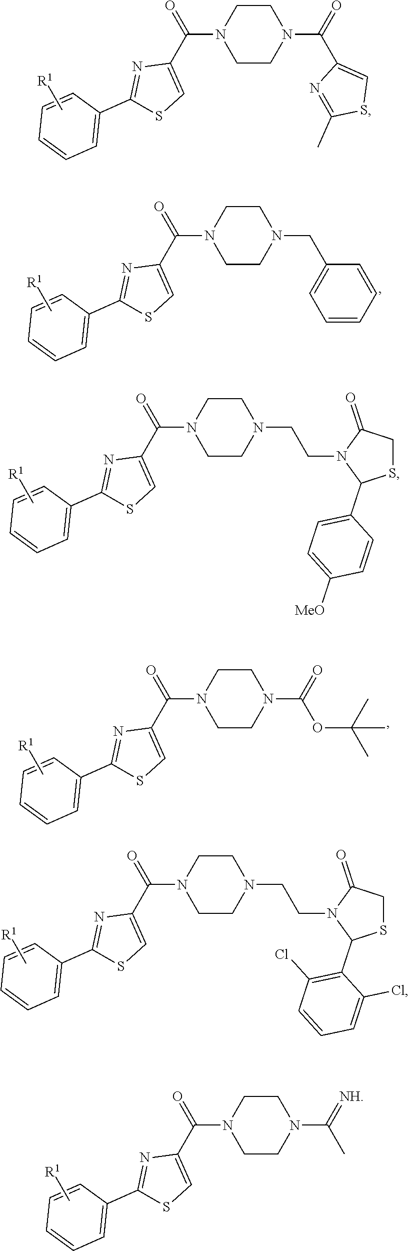

In one embodiment, the compound is represented by general Formula Ib:

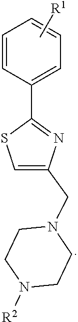

In one embodiment, R¹ represents a substituent selected from the group consisting of: a halogen atom (such as fluorine, chlorine, bromine or iodine), an alkyloxycarbonyl (such as tert-butyl-, ethyl-, butyl-, propyl-, iso-propyl-, and methyl-oxycarbonyl), a hydrogen atom, an alkoxy group (such as methoxy, an ethoxy, tert-butoxy), and a C₁-C₅ alkyl group, or any combination thereof.

In one embodiment, the compound comprises at least one of:

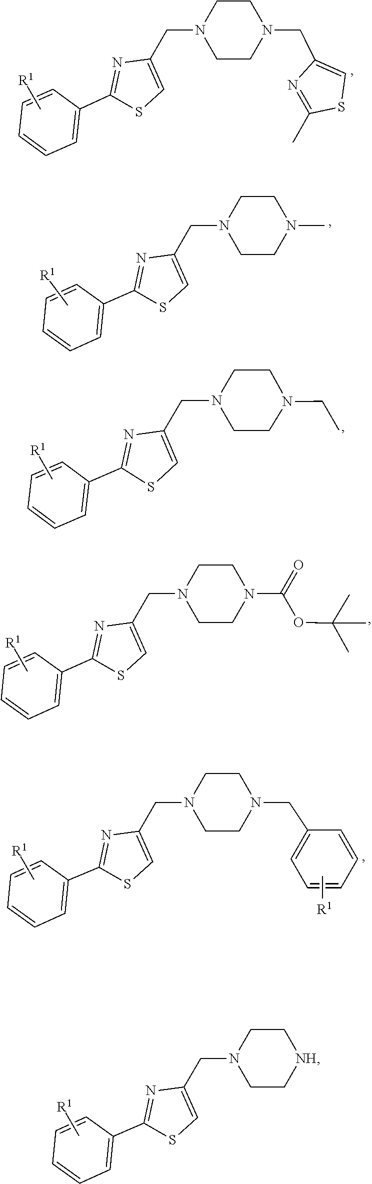

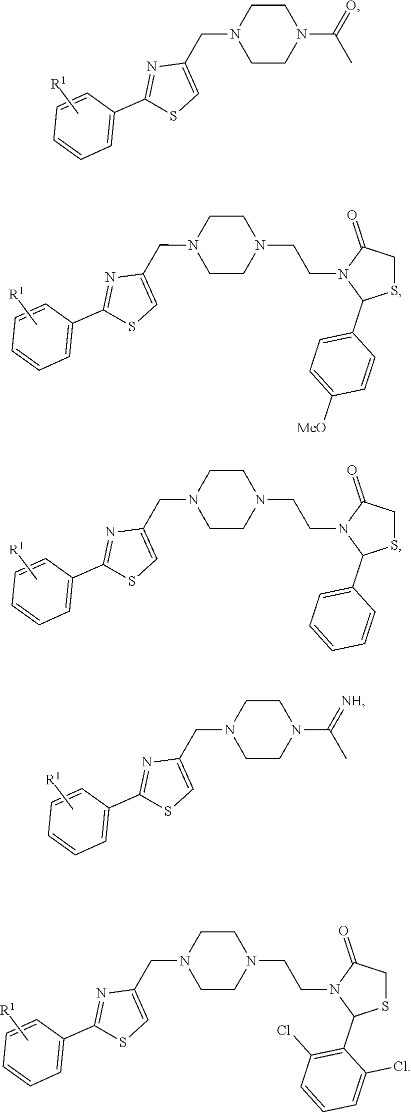

In one embodiment, the compound is represented by general Formula II:

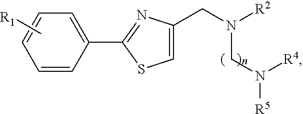

wherein: R² is selected from a hydrogen atom, an alkyl, or is absent; R⁴ and R⁵ are each independently selected from the group consisting of: a hydrogen atom, a straight or branched and unsubstituted or substituted C₁-C₁₂ alkyl, aralkyl, aryl, heteroaryl group, and any combination thereof, and n is the number of carbons of a straight or branched and unsubstituted or substituted carbon chain ranging from 1 to 10.

In one embodiment, the compound is represented by general Formula IIa:

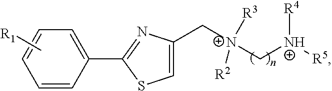

wherein each of R² and R³ is independently selected from a hydrogen atom, an alkyl, or is absent.

In one embodiment, R¹ represents a substituent selected from the group consisting of: a halogen atom (such as fluorine, chlorine, bromine or iodine), an alkyloxycarbonyl (such as tert-butyl-, ethyl-, butyl-, propyl-, iso-propyl-, and methyl-oxycarbonyl), a hydrogen atom, an alkoxy group (such as methoxy, an ethoxy, tert-butoxy), and a C₁-C₅ alkyl group, or any combination thereof, and wherein n is from 1 to 5.

In one embodiment, the compound is represented by general Formula III:

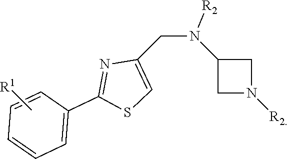

In one embodiment, the compound is represented by general Formula IIIa:

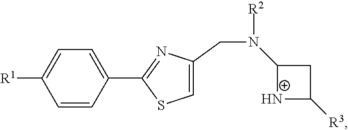

wherein R¹ represents a substituent selected from the group consisting of: a halogen atom (such as fluorine, chlorine, bromine or iodine), an alkyloxycarbonyl (such as tert-butyl-, ethyl-, butyl-, propyl-, iso-propyl-, and methyl-oxycarbonyl), a hydrogen atom, an alkoxy group (such as methoxy, an ethoxy, tert-butoxy), and a C₁-C₅ alkyl group, or any combination thereof; and wherein each of R² and R³ is independently selected from a hydrogen atom, an alkyl, or is absent.

In one embodiment, the compound is represented by general Formula IV:

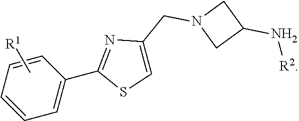

In one embodiment, the compound comprises:

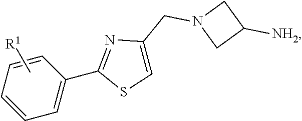

wherein R¹ represents a substituent selected from the group consisting of: a halogen atom (such as fluorine, chlorine, bromine or iodine), an alkyloxycarbonyl (such as tert-butyl-, ethyl-, butyl-, propyl-, iso-propyl-, and methyl-oxycarbonyl), a hydrogen atom, an alkoxy group (such as methoxy, an ethoxy, tert-butoxy), and a C₁-C₅ alkyl group, or any combination thereof.

In one embodiment, the compound or the salt thereof comprises at least one protonated nitrogen atom.

In one embodiment, the compound or the salt thereof has an IC₅₀ ranging from 1-500 μM.

In another aspect, there is provided a pharmaceutical composition comprising the antibacterial composition of the invention and a pharmaceutically acceptable carrier, for use in reducing or inhibiting bacterial growth.

In another aspect, there is provided an article coated with the antibacterial composition of the invention.

In another aspect, there is provided a method of inhibiting or reducing bacterial growth, comprising contacting the bacteria with the antibacterial composition of the invention.

In another aspect, there is provided a method of treating a bacterial infectious disease or a symptom thereof in a subject in need thereof, comprising administering to the subject a therapeutically effective amount of a pharmaceutical composition of the invention, thereby treating or inhibiting the bacterial infectious disease or a symptom thereof in the subject.

In one embodiment, reducing bacterial growth comprises reducing one or more bacterial cellular activities selected from the group consisting of: cellular replication rate, number of replicating cells, protein synthesis rate, peptidyl transferase center (PTC) activity, and yields of protein synthesis.

In one embodiment, the bacterium is selected from the group consisting of Mycobacterium, and Staphylococcus.

In one embodiment, the Mycobacterium is Mycobacterium tuberculosis.

In one embodiment, the Staphylococcus is Staphylococcus aureus.

In one embodiment, the bacteria comprise a drug-resistant bacterium.

In one embodiment, the drug-resistant bacterium is resistant to one or more types of drugs.

In one embodiment, the drug is an antibiotic drug.

In another aspect of the invention, there is provided a method for identifying a compound which binds to a biological target, comprising: a. performing a T2 relaxation NMR by to a sample comprising a biological target and a fragment molecule, thereby determining a lead fragment having binding affinity to the biological target; b. generating a dataset comprising a plurality of virtual molecules, wherein the virtual molecules comprise a fragment having at least 70% homology to the lead fragment; c. performing high throughput docking on the dataset to obtain values of binding energy (ΔG) with respect to the biological target for each of the virtual molecules, thereby identifying the compound.

In another aspect of the invention, there is provided a system comprising: at least one hardware processor; and a non-transitory computer-readable storage medium having stored thereon program instructions, the program instructions executable by the at least one hardware processor to: receive, as input, a dataset comprising a plurality of virtual molecules and corresponding values of binding energy (ΔG) with respect to a biological target; calculating a plurality of features for each of the virtual molecules; at a training stage, train a machine learning (ML) model on a training set comprising: a. the plurality of features, and b. labels associated with the corresponding values of ΔG; at an inference stage, apply the ML model to a new virtual molecule to predict a ΔG value with respect to the biological target.

In one embodiment, at the training stage each of the virtual molecules is labeled with the labels associated with the corresponding values of ΔG.

In one embodiment, the plurality of features comprises a variable selected form the group consisting of: DIST, MOD and VAR or any combination thereof.

In one embodiment, the variable is associated with coordinates of each atom belonging to a lead fragment of the virtual molecule, wherein the lead fragment is identified by T2 relaxation NMR.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1. plan for development of small molecule inhibitors for ribosomal PTC. The approach combines NMR-fragment based screening with virtual screening. Using NMR (T₂ relaxation) and a fragment library, scaffolds that bind hairpin 91 in the ribosomal PTC of Mtb were identified (2). These scaffolds were used to filter larger compounds containing the fragment molecules from the ZINC database (3). Nearly thousand compounds containing phenyl thiazole were docked, using Autodock software, into the PTC of the available crystal structure of a bacterial ribosome, and hits were ranked on the basis of the binding energy (4). Conclusions as to structure activity relationships were drawn using machine learning (ML) algorithms, and ten compounds were selected and tested for their ability to inhibit translation in Mtb (5).

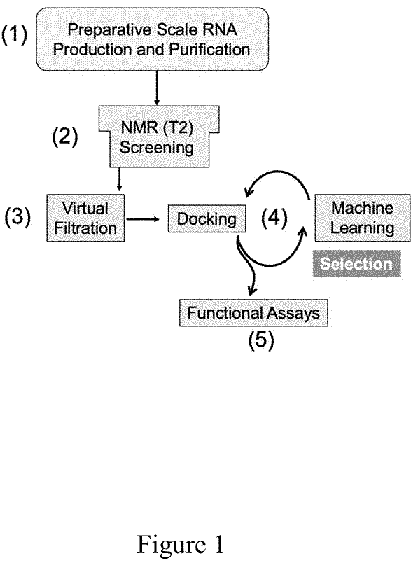

FIGS. 2A-B exhibit RNA molecules used in this study. FIG. 2A depicts the sequence SEQ ID NO: 2 (GCAUCCUGGGGCUGUAGUCGGUCCCAAGG) of the S. aureus RNA hairpin. The modified nucleotides in the sequence (SEQ ID NO: 1) of M. tuberculosis used for T2 relaxation screening, are marked in arrows. A superposed hairpin 91 structure from the ribosomal PTC of M. smegmatis is shown in grey. Root mean square (RMS) values for PTC and hairpin 91 of S. aureus and M. smegmatis are 0.858 and 0.788, respectively. FIG. 2B represents a graph showing thermal stability of PTC-RNA from S. aureus compared to that of Mtb. The thermal stability of RNA sequences was measured by circular dichroism spectroscopy.

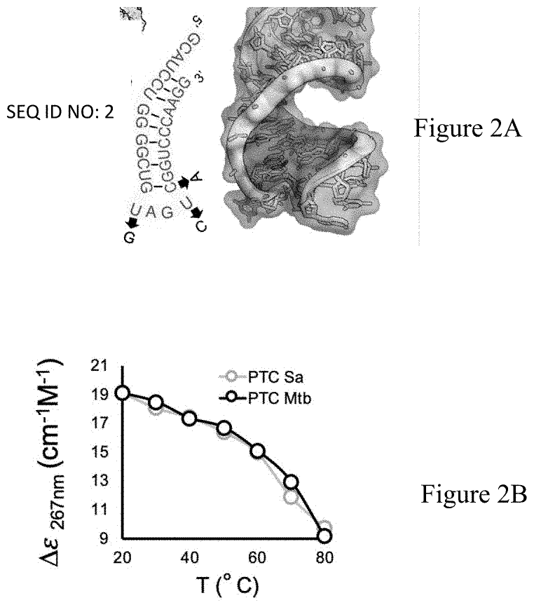

FIG. 3 shows identification of N-methyl-4-(1,3-thiazol-2-yl)benzylamine hits from a T₂-edited (CPMG NMR) mini-screen. The data is from pool number 2 of three pools consisting of 10 fragments each, with and without the target RNA. All spectra are expanded to show the aromatic region. CPMG T₂-edited spectra with CPMG delay times as indicated on the figure for spectra with (red) and without (blue) the target RNA.

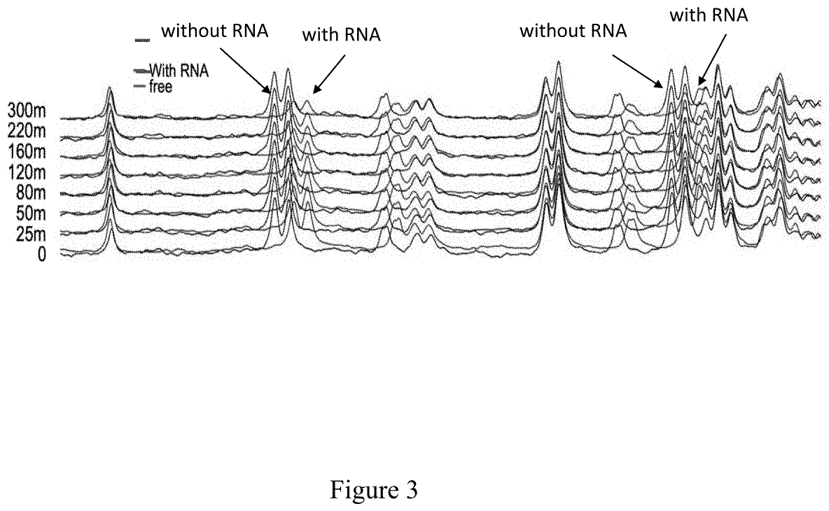

FIG. 4 shows the effect of compounds on protein synthesis measured by in vitro translation reaction. The effect of the compounds was tested against M. smegmatis ribosomes. Inhibition of mycobacterial growth by compound Nr. 2, 1-[(2-methylthiazol-4-yl)methyl]-4-[(2-phenylthiazol-4-yl)methyl]piperazine, and compound Nr. 8, 1-ethyl-4-[(2-phenylthiazol-4-yl)methyl]piperazine), in the indicated concentrations is presented. The IC₅₀ values for compound Nr. 2 and compound Nr. 8 are 8.9 and 4.8 μM, respectively. The IC₅₀ value of chloramphenicol was found to be 9.2 μM.

Luciferase activity was measured as a matter of compound concentration. Compound Nr. 2 ( [symbol] ) was found to be as effective inhibitor of luciferase activity as chloramphenicol (CAP; ●), while compound Nr. 8 (∘) was found to about twice as better than both.

FIG. 5 shows in-line probing gel image of ribosomal PTC of Mtb. The RNA segment used for this experiment (SEQ ID NO: 1) was in-vitro transcribed and purified. The reaction contained 5 nM RNA labeled with ³²P at its 5′ terminus and in-line reaction buffer: 50 mM Tris-HCl pH 8.3, 20 mM MgCl₂, and 100 mM KCl. After incubation at 25° C. for 50 hours, RNA product was analyzed by electrophoresis through 10% 29:1 polyacrylamide gel containing 8 M urea and visualized using autoradiography. a, Lane 1-4 contains the untreated RNA (C1), partially digested using T1 RNase (T1), alkaline digested (—OH, C2). Lane 4-5 shows cleavage pattern of the RNA in the absence of the small molecules (C3) and in the presence of DMSO (D). Lanes 6-9 and lanes 10-13 contain compound Nr. 2 and compound Nr. 8, respectively, in the indicated concentrations. b, secondary structure model of the RNA present hotspots of scission in the presence of the compounds.

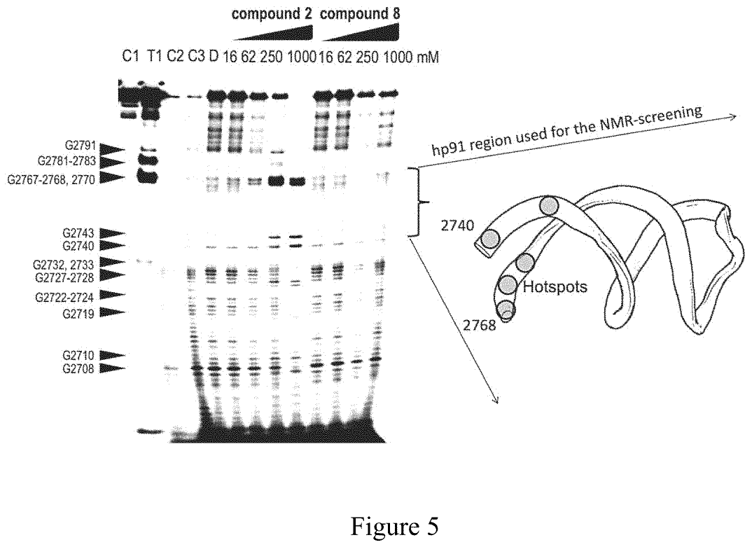

FIG. 6 shows ML model-based algorithm for SAR. Each location is numbered and its influence on the value of the binding energy is marked with a color: red—very significant, yellow—medium significance, teal—low significance and grey not significant. b. Projection of 400 33-dimensional data points on the top two principal components of the data matrix. The 400 data points correspond to the first and fourth quartile in terms of binding energy. Blue points have the strongest binding energy and red the weakest. Clear separation can be observed between most of the blue and red points.

FIGS. 7A-B show T2 analysis by CPMGNMR. FIG. 7A shows the intensity of the proton peaks from 1D CPMG spectra. The data is from pool 81 containing fragment molecules with and without the target RNA. FIG. 7A shows T2 analysis of peaks 1-3, originating from one molecule, [2-(3-chlorophenyl)-1,3-thiazol-4-yl]methanamine, which shows the largest change in T2 upon addition of RNA. FIG. 7B shows T2 analysis of peaks 4-6, originating from different molecules in the same pool that do not show any significant change in T2 upon addition of RNA.

### DETAILED DESCRIPTION OF THE INVENTION

The present invention, in some embodiments thereof, is directed to antibacterial compounds and compositions comprising thereof.

### Compounds

In one aspect of the invention, there is provided a composition comprising a compound or a salt thereof, wherein the compound is represented by general Formula A:

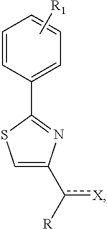

wherein R is selected from the group consisting of:

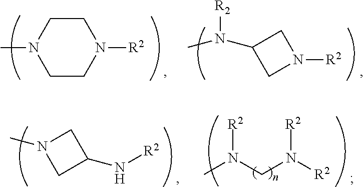

each R² is independently selected from the group consisting of: a hydrogen atom, a linear or branched, and unsubstituted or substituted C₁-C₁₂ alkyl, aralkyl, aryl, carboxy group, imine, heteroaryl group, benzyl, a heterocyclyl, an alkyloxycarbonyl (such as tert-butyl-, ethyl-, butyl-, propyl-, iso-propyl-, and methyl-oxycarbonyl), an alkoxy group (such as methoxy, an ethoxy, tert-butoxy), or any combination thereof,
X comprises an atom selected from the group consisting of: hydrogen, carbon, oxygen, nitrogen, and sulfur or any combination thereof, [symbol]
represents a double bond or a single bond; n represents an integer being in a range between 1 and 40; and R¹ represents a substituent selected from the group consisting of: a halogen atom, hydrogen, a nitro group, an amine group, an azide group, a cyano group, a methoxy group, a carboxylic group, an ester group, an ether group, an aromatic group, alkyl group, vinyl group, an alcohol, a carbamate group, a thiazole group, an urea group, or any combination thereof.

In some embodiments, R¹ represents a substituent in para position.

In some embodiments, n represents an integer being in a range between 1 and 40, 1 and 30, 1 and 35, 1 and 20, 1 and 25, 1 and 15, 1 and 10, 1 and 5, 1 and 3, including any range or value therebetween.

In some embodiments, the compound is represented by general Formula I:

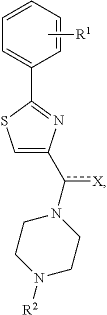

wherein X, R¹ and R² are as described hereinabove.

In some embodiments, X comprises an atom selected from the group consisting of: hydrogen, carbon, and oxygen, or any combination thereof. In some embodiments, X is selected from the group consisting of: hydrogen, —O, —OH, —CH₂, —CH₃, —NH, —NH₂, —S, and —SH or any combination thereof. In some embodiments, X is any of hydrogen, —O, —OH, or a combination thereof. In some embodiments, X is O or NH.

In some embodiments, the compound is represented by general Formula Ia:

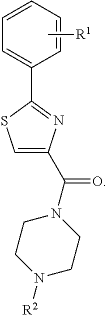

In some embodiments, R¹ represents a substituent selected from the group consisting of: a halogen atom (such as fluorine, chlorine, bromine or iodine), a hydrogen atom, an alkoxy group (such as methoxy, an ethoxy, tert-butoxy), a hydroxy group, and an alkyl group, or any combination thereof. In some embodiments, R¹ represents a para-substituent.

In some embodiments, R¹ is selected from the group consisting of: a halogen atom (such as fluorine, chlorine, bromine or iodine), a hydrogen atom, an alkoxy group (such as methoxy, an ethoxy, tert-butoxy), and a C₁-C₅ alkyl group, or any combination thereof.

In some embodiments, R² is selected from the group consisting of: a hydrogen atom, a linear or branched, and unsubstituted or substituted C₁-C₅ alkyl, aryl, carboxy group, imine, heteroaryl group, benzyl, a heterocyclyl, an alkyloxycarbonyl (such as tert-butyl-, ethyl-, butyl-, propyl-, iso-propyl-, and methyl-oxycarbonyl), an alkoxy group (such as methoxy, an ethoxy, tert-butoxy), or any combination thereof.

In some embodiments, the compound comprises at least one of:

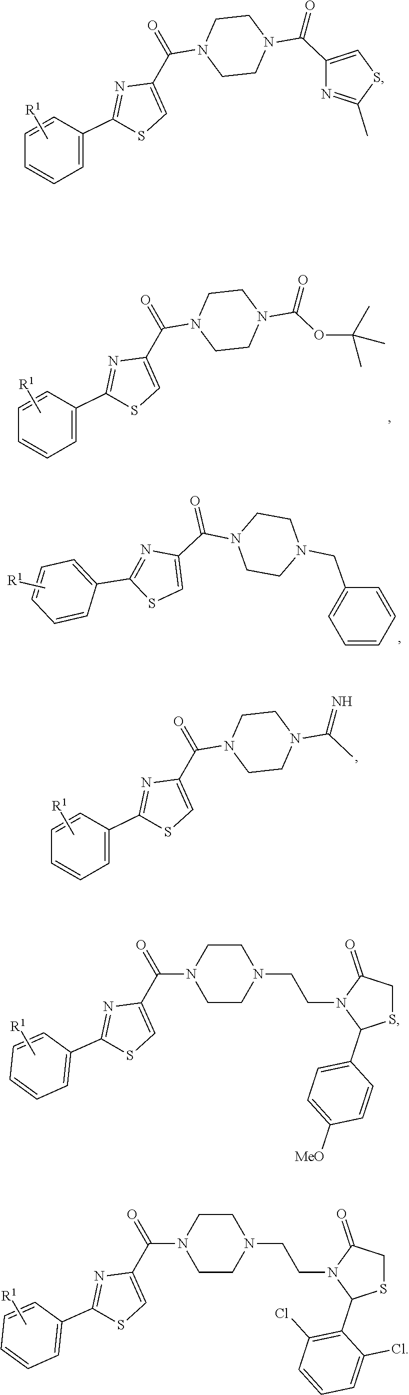

In some embodiments, R¹ is selected from the group consisting of: a halogen atom (such as fluorine, chlorine, bromine or iodine), a hydrogen atom, and a C₁-C₅ alkyl group, or any combination thereof. In some embodiments, R¹ is a hydrogen atom. In some embodiments, R¹ is a halogen atom. In some embodiments, R¹ is a C₁-C₅ alkyl group linear or branched.

In some embodiments, the compound or the salt thereof is represented by general Formula Ib:

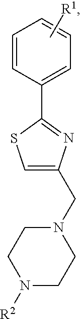

or by general Formula IB:

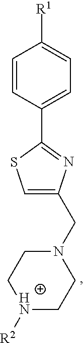

wherein R¹ and R² are as described hereinabove.

In some embodiments, the compound is represented by general Formula Ic:

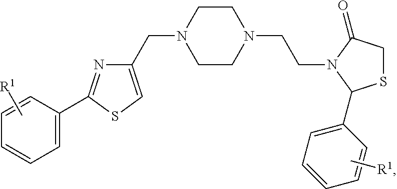

wherein R¹ is as described hereinabove.

In some embodiments, the compound is represented by general Formula Id:

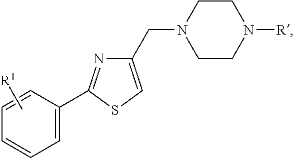

wherein R¹ is as described hereinabove and R′ is a linear or branched, and unsubstituted or substituted C₁-C₅ alkyl, aryl, benzyl, hydrogen and an alkyloxycarbonyl (such as butyl-, tert-butyl-, ethyl-, propyl-, iso-propyl-, and methyl-oxycarbonyl), or any combination thereof.

In some embodiments, the compound is any one of:

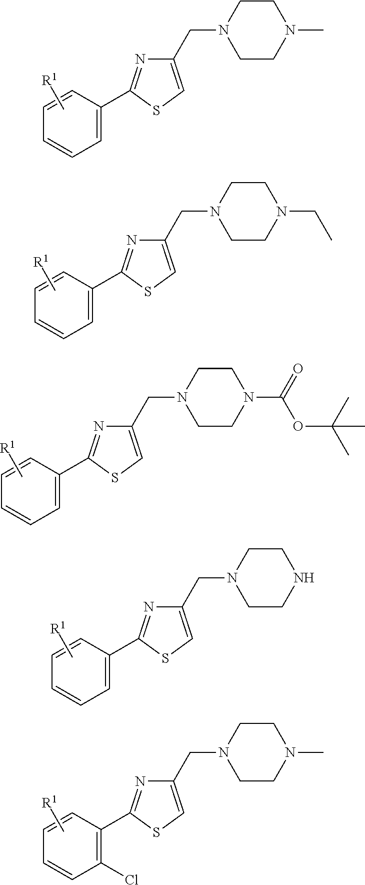

wherein R¹ is any of a linear or branched, and unsubstituted or substituted C₁-C₅ alkyl, a halogen atom and hydrogen, or any combination thereof. In some embodiments, R¹ is a para-substituent.

In some embodiments, the compound is any one of:

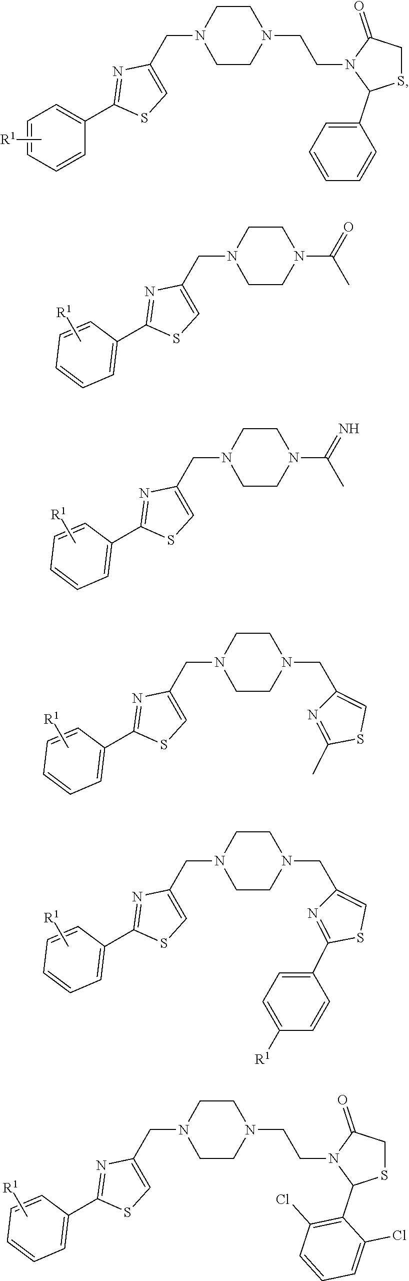

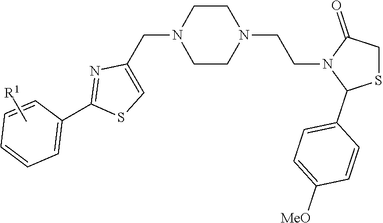

In some embodiments, the compound is represented by general Formula Ie:

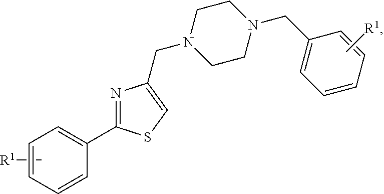

wherein R¹ is as described hereinabove.

In some embodiments, R¹ is selected from the group consisting of: a halogen atom (such as fluorine, chlorine, bromine or iodine), a hydrogen atom, a C₁-C₅ alkoxy group, and a C₁-C₅ alkyl group, or any combination thereof. In some embodiments, R¹ is a hydrogen atom. In some embodiments, R¹ is a halogen atom. In some embodiments, R¹ is a C₁-C₅ alkyl group linear or branched. In some embodiments, R¹ is methoxy.

In another aspect of the invention, the compound is represented by general Formula II:

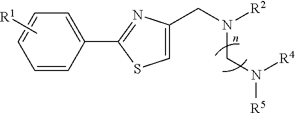

wherein R² is selected from a hydrogen atom, an alkyl, or is absent; R⁴ and R⁵ are each independently selected from the group consisting of: a hydrogen atom, a straight or branched and unsubstituted or substituted C₁-C₁₂ alkyl, aralkyl, aryl, heteroaryl group, and any combination thereof, n is the number of carbons of a straight or branched and unsubstituted or substituted carbon chain ranging from 1 to 10; and wherein R¹ is as described hereinabove.

In some embodiments, R¹ represents a substituent in para position.

In some embodiments, the compound or the salt thereof is represented by general Formula IIa:

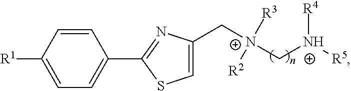

wherein each of R² and R³ is independently selected from a hydrogen atom, an alkyl, or is absent.

In some embodiments, R¹ represents a substituent selected from the group consisting of: a halogen atom (such as fluorine, chlorine, bromine or iodine), an alkyloxycarbonyl (such as tert-butyl-, ethyl-, butyl-, propyl-, iso-propyl-, and methyl-oxycarbonyl), a hydrogen atom, an alkoxy group (such as methoxy, an ethoxy, tert-butoxy), and a C₁-C₅ alkyl group, or any combination thereof, and wherein n is from 1 to 5. In some embodiments, R¹ comprises a halogen atom, a C₁-C₅ alkyl group, a hydrogen atom, or a combination thereof.

In another aspect of the invention, the compound is represented by general Formula III:

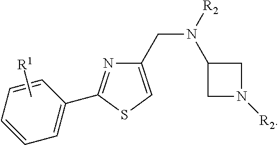

In some embodiments, the compound or the salt thereof is represented by general Formula IIIa:

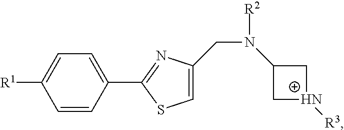

wherein R¹ is as described hereinabove, and wherein each of R² and R³ is independently selected from a hydrogen atom, an alkyl, or is absent.

In some embodiments, R¹ comprises a halogen atom, a C₁-C₅ alkyl group, a hydrogen atom, or a combination thereof.

In some embodiments, each of R² and R³ is independently selected from a hydrogen atom, a C₁-C₅ alkyl group, or is absent.

In some embodiments, the compound is represented by general Formula IV:

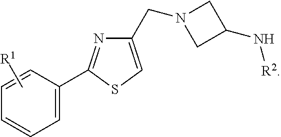

In some embodiments, the compound comprises

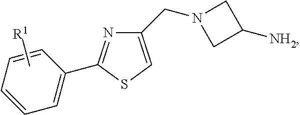

wherein R¹ is as described hereinabove.

In some embodiments, R¹ is a halogen, hydrogen, or C₁-C₃ alkyl. In some embodiments, R¹ is an ortho- or a para-substituent. In some embodiments, R¹ is a methyl group in a para-position. In some embodiments, R¹ is a chloro group in ortho-position. In some embodiments, R¹ is a hydrogen atom.

In some embodiments, the compound or the salt thereof comprises at least one protonated nitrogen atom.

In some embodiments, the compound or the salt thereof is any one of.

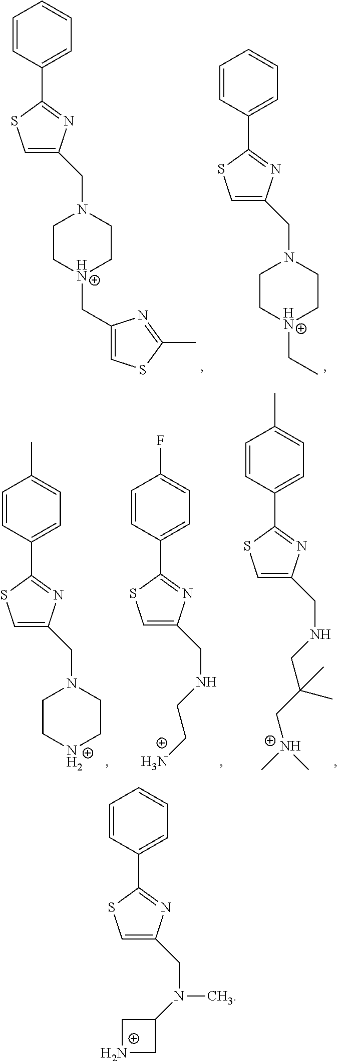

In some embodiments, the compound is:

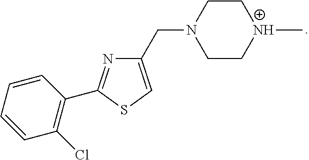 [SMILES: C[NH+]1CCN(Cc2csc(-c3ccccc3Cl)n2)CC1]

In some embodiments, the compound of the invention is chemically synthesized or biosynthesized. Methods of biosynthesis are well known within the art, and can include, but are not limited to: production in a cell culture or enzymatic cell-free production. In some embodiments, the cell culture is a mono- or poly-culture.

As used herein, the term “alkyl” describes an aliphatic hydrocarbon including straight chain and branched chain groups. In some embodiments, the alkyl group has 4 to 100 carbon atoms, and in some embodiments, 4-40 carbon atoms. Whenever a numerical range; e.g., “4-100”, is stated herein, it implies that the group, in this case the alkyl group, may contain 4 carbon atoms, 5 carbon atoms, 6 carbon atoms, etc., up to and including 100 carbon atoms. In the context of the present invention, a “long alkyl” is an alkyl having at least 20 carbon atoms in its main chain (the longest path of continuous covalently attached atoms). A short alkyl therefore has 20 or less main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings which share an adjacent pair of carbon atoms) group where one or more of the rings does not have a completely conjugated pi-electron (π-electron) system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an —O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, alkoxy, nitro, amine, hydroxyl, thiol, thioalkoxy, thiohydroxy, carboxy, amide, aryl and aryloxy, depending on the substituted group and its position in the molecule. Additional substituents are also contemplated.

The term “halide”, “halogen” or “halo” describes fluorine, chlorine, bromine or iodine.

The term “haloalkyl” describes an alkyl group as defined herein, further substituted by one or more halide(s).

The term “haloalkoxy” describes an alkoxy group as defined herein, further substituted by one or more halide(s).

The term “hydroxyl” or “hydroxy” describes a —OH group.

The term “thiohydroxy” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amine” describes a —NR′R″ group, with R′ and R″ as described herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

The term “carboxy” or “carboxylate” describes a —C(═O)—OR′ group, where R′ is hydrogen, alkyl, cycloalkyl, alkenyl, aryl, heteroaryl (bonded through a ring carbon) or heteroalicyclic (bonded through a ring carbon) as defined herein.

The term “carbonyl” describes a —C(═O)—R′ group, where R′ is as defined hereinabove.

The above-terms also encompass thio-derivatives thereof (thiocarboxy and thiocarbonyl).

The term “thiocarbonyl” describes a —C(═S)—R′ group, where R′ is as defined hereinabove.

A “thiocarboxy” group describes a —C(═S)—OR′ group, where R′ is as defined herein.

A “carbamyl” or “carbamate” group describes an —OC(═O)—NR′R″ group, where R′ is as defined herein and R″ is as defined for R′.

A “nitro” group refers to a —NO₂ group.

A “cyano” or “nitrile” group refers to a —C≡N group.

As used herein, the term “azide” refers to a —N₃ group.

The term “alkaryl” describes an alkyl, as defined herein, which substituted by an aryl, as described herein. An exemplary alkaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

As used herein, the terms “halo” and “halide”, which are referred to herein interchangeably, describe an atom of a halogen, that is fluorine, chlorine, bromine or iodine, also referred to herein as fluoride, chloride, bromide and iodide.

The term “haloalkyl” describes an alkyl group as defined above, further substituted by one or more halide(s).

### Compositions and Articles

In some embodiments, the invention is directed to a composition comprising one or more compounds, wherein the compounds are as described hereinabove. In some embodiments, one or more compounds are represented by any one of general Formulae I, Ia-e, II, IIa, III, IIIa, and IV.

In some embodiments, the composition of the invention further comprises at least one acceptable carrier or diluent.

In some embodiments, terms “composition”, “pharmaceutical composition” and “antibacterial composition” are used herein interchangeably.

In some embodiments, the composition or the kit of the invention is for use in bacterial infection therapy. In some embodiments, the composition or the kit is an antibacterial composition or kit, wherein the compound or the salt thereof has a half maximal inhibitory concentration (IC₅₀) ranging from 1-1,200 μM.

In some embodiments, the composition or the kit of the invention inhibits bacterial PTC. In some embodiments, the composition or the kit of the invention is for inhibiting bacterial PTC. In some embodiments, the composition or the kit is for inhibiting bacterial PTC in a selective manner. In some embodiments, the composition or the kit is for inhibiting bacterial PTC in a subject in need thereof. In some embodiments, the subject is a human subject or a bacterium. In some embodiments, the composition or the kit is for inhibiting Staphylococcal (e.g. S. aureus) PTC. In some embodiments, the composition of the invention inhibits Staphylococcal (e.g. S. aureus) PTC. In some embodiments, inhibition comprises at least 50%, at least 60%, at least 65%, at least 70%, at least 75%, at least 80%, at least 90%, at least 95%, at least 97% of the enzymatic activity of PTC. In some embodiments, inhibition is a selective inhibition of the bacterial PTC. In some embodiments, inhibition is a competitive or a non-competitive inhibition. In some embodiments, the inhibition is reversible.

In some embodiments, inhibiting enzymatic activity of PTC results in inhibiting or reducing ribosomal activity. In some embodiments, ribosomal activity comprises any of: bacterial protein production rate, bacterial protein production yield, bacterial protein synthesis, bacterial messenger RNA translation rate or any combination thereof. In some embodiments, ribosomal activity is related to synthesis of a peptide bond within the ribosome.

In some embodiments, the composition or the kit of the invention inhibits Mycobacterial (e.g. M. smegmatis, M tuberculosis) PTC. In some embodiments, the composition or the kit is for inhibiting Mycobacterial (e.g. M. smegmatis, M. tuberculosis) PTC. In some embodiments, the composition, the kit, the antibacterial composition or the pharmaceutical composition has a half maximal inhibitory concentration (IC₅₀) with respect to bacterial PTC ranging from 1-1,200 μM, 1-10 μM, 10-20 μM, 1-20 μM, 1-15 μM, 20-30 μM, 5-20 μM, 10-30 μM, 20-40 μM, 25-50 μM, 30-60 μM, 40-70 μM, 50-80 μM, 65-90 μM, 70-100 μM, 80-110 μM, 90-120 μM, 110-160 μM, 150-275 μM, 250-500 μM, 400-700 μM, 650-850 μM, 800-1,000 μM, 1000-1,200 μM including any range or value therebetween.

According to some embodiments, there is provided an article coated with a compound of the invention. In some embodiments, the article is coated with one or more compounds of the invention. In some embodiments, the article is coated with a composition comprising a compound having an antibacterial activity, wherein the compound is represented by any one of the structural general formulae I, Ia-e, II, IIa, III, IIIa, and IV.

In some embodiments, the invention is directed to a composition comprising as an active ingredient an effective amount of the compound of the invention, and an acceptable carrier and/or diluent. In some embodiments, the invention is directed to a composition comprising as an active ingredient an effective amount of one or more compounds represented by the structural general formulae I, Ia-e, II, IIa, III, IIIa, and IV, or a combination thereof. In some embodiments, the acceptable carrier facilitates incorporation or coating of the active ingredient(s) to a substrate.

In some embodiments, a composition, further comprises a substrate. In some embodiments a composition comprising one or more compounds represented by the structural general formulae I, Ia-e, II, IIa, III, IIIa, and IV or a combination thereof, is incorporated in and/or on at least a portion of the substrate.

In some embodiments, the invention is directed to a composition comprising a substrate having incorporated in and/or on at least a portion thereof, one or more compounds represented by the structural general formulae I, Ia-e, II, IIa, III, IIIa, and IV or a combination thereof. As used herein, the term “a portion thereof” refers to, for example, a surface or a portion thereof, and/or a body or a portion thereof, of solid or semi-solid substrates; or a volume or a part thereof, of liquid, gel, foams and other non-solid substrates.

Substrates of widely different chemical nature can be successfully utilized for incorporating (e.g., depositing on a surface thereof) one or more compounds represented by the structural general formulae I, Ia-e, II, IIa, III, IIIa, and IV or a combination thereof, or a composition comprising thereof, thereon, as described herein. The term “successfully utilized” refers to an outcome meaning that: (i) one or more compounds of the invention, or a composition comprising thereof, successfully formed a uniform and homogenously coating on the substrate's surface; and (ii) the resulting coating imparts long-lasting desired properties (e.g., antimicrobial properties) to the substrate's surface.

Substrate usable according to some embodiments of the present invention can therefore be hard (rigid) or soft, solid, semi-solid, or liquid substrates, and may take a form of a foam, a solution, an emulsion, a lotion, a gel, a cream or any mixture thereof.

Substrate usable according to some embodiments of the present invention can have, for example, organic or inorganic surfaces, including, but not limited to, glass surfaces; porcelain surfaces; ceramic surfaces; silicon or organosilicon surfaces, metallic surfaces (e.g., stainless steel); mica, polymeric surfaces such as, for example, plastic surfaces, rubbery surfaces, paper, wood, polymer, a metal, carbon, a biopolymer, silicon mineral (rock or glass), surfaces, wool, silk, cotton, hemp, leather, fur, feather, skin (hide, pelt or pelage) surfaces, plastic surfaces and surfaces comprising or made of polymers such as but not limited to polypropylene (PP), polycarbonate (PC), polyethylene (PET), high-density polyethylene (HDPE), low-density polyethylene (LDPE), polyester (PE), unplasticized polyvinyl chloride (PVC), and fluoropolymers including but not limited to polytetrafluoroethylene (PTFE, Teflon); or can comprise or be made of any of the foregoing substances, or any mixture thereof.

Alternatively, other portions, or the entire substrate are made of the above-mentioned materials.

In some embodiments, the substrate incorporating one or more compounds of the invention, or a composition comprising thereof, as described herein is or forms a part of an article.

According to some embodiments, an article (e.g., an article-of-manufacturing) comprises a substrate incorporating in and/or on at least a portion thereof one or more compounds of the invention or a composition comprising thereof.

The article can be any article which can benefit from the antibacterial activities of one or more compounds of the invention or a combination thereof.

Non-limiting examples of articles include, but are not limited to, medical devices, organic waste processing device, fluidic device, an agricultural device, a package, a sealing article, a fuel container, a water and cooling system device and a construction element.

Non-limiting examples of devices which can incorporate at least one of one or more compounds of the invention or a combination thereof, or a composition comprising thereof, as described herein, beneficially, include tubing, pumps, drain or waste pipes, screw plates, and the like.

Non-limiting example of an article include but is not limited to an element used in water treatment systems (such as for containing and/or transporting and/or treating aqueous media or water), devices, containers, filters, tubes, solutions and gases and the likes.

Non-limiting example of an article include but is not limited to an element in organic waste treatment systems (such as for containing and/or disposing and/or transporting and/or treating organic waste), devices, containers, filters, tubes, solutions and gases and the likes.

In some embodiments, the invention is directed to a pharmaceutical composition comprising as an active ingredient a therapeutically effective amount of the compound of the invention, and a pharmaceutically acceptable carrier and/or diluent. In some embodiments, the invention is directed to a pharmaceutical composition comprising as an active ingredient a therapeutically effective amount of one or more compounds of the invention or any combination thereof. In some embodiments, the pharmaceutically acceptable carrier facilitates administration of the compound of the invention to an organism. For example, the term “pharmaceutically acceptable” can mean approved by a regulatory agency of the Federal or a state government or listed in the U.S. Pharmacopeia or other generally recognized pharmacopeia for use in animals, and more particularly in humans.

In some embodiments, the disclosed invention is directed to a composition for use in inhibiting, reducing or decreasing microbial activity, or combination thereof. In some embodiments, microbial activity comprises any activity selected from the group consisting of: proliferation, antibiotic resistance, cell communication or quorum sensing, biofilm production, toxin production or secretion, and combination thereof. Microbial activity can be assayed using any common method, non-limiting examples of which include, but are not limited to, spectrophotometry, drug resistance assays using selective substrates, bioluminescence assay, liquid chromatography and mass-spectrometry or others, which would be apparent to one of ordinary skill in art.

In some embodiments, the disclosed invention is directed to a composition for use in inhibiting, reducing or decreasing bacterial load in a subject in need thereof. In some embodiments, the composition is for use in inhibiting, reducing or decreasing microbial activity in a subject in need thereof. In some embodiments, the microbial activity is related to an infection by one or more bacterial species. In some embodiments, the composition is for use in inhibiting, reducing or decreasing microbial activity related to Staphylococcus aureus infection. In some embodiments, the microbial activity is related to a ribosomal activity. In some embodiments, the ribosomal activity comprises PTC activity.

As used herein, the term “anti-microbial activity” refers to the ability to inhibit, prevent, reduce or retard any one of: bacterial growth, bacterial protein production rate, bacterial protein production yield, bacterial protein synthesis, bacterial messenger RNA translation rate, bacterial RNA stability, biofilm formation, or eradicate living bacterial cells, or their spores. In some embodiments, anti-microbial activity is achieved on a surface or in a moist environment. In some embodiments, inhibiting or reducing or retarding the formation of load of a microorganism refers to inhibiting, reducing or retarding growth of microorganisms and/or eradicating a portion or all of an existing population of microorganisms. The terms “anti-microbial” and “anti-bacterial” are used herein are interchangeably.

In some embodiments, reduced, inhibited or retarded bacterial mRNA translation rate, bacterial protein synthesis, bacterial protein production rate, bacterial protein production yield, or any combination thereof, is indicative of reduced, inhibited or retarded bacterial fitness. As used herein, “bacterial fitness” encompasses any bacterial capability that is required for sustaining life, such as: DNA replication, cell division, cell growth, wherein growth comprises size, and volume, and the like.

Methods for determining reduced protein synthesis, protein production rates, and yields, are common and would be apparent to one of ordinary skill in the art, non-limiting example of which is shown hereinbelow, using a Luciferase reporter assay.

In some embodiments, inhibit, prevent, retard, or reduce is by at least 5%, at least 15%, at least 25%, at least 40%, at least 50%, at least 65%, at least 75%, at least 85%, at least 90%, at least 95%, at least 99% compared to control, or any value or range therebetween. In some embodiments, modifying is by 1-5%, 7-15%, 10-25%, 20-40%, 35-50%, 45-65%, 55-75%, 70-85%, 80-90%, 87-95%, or 92-100% compared to control. Each possibility represents a separate embodiment of the invention.

In some embodiments, a compound of the invention is present in a composition at a concentration of at least 1 μM, at least 2 μM, at least 5 μM, at least 10 μM, at least 15 μM, at least 20 μM, at least 30 μM, at least 40 μM, at least 50 μM, at least 75 μM, at least 100 μM, at least 150 μM, at least 200 μM, at least 225 μM, at least 350 μM, at least 400 μM, at least 500 μM, at least 600 μM, at least 750 μM, at least 1,000 μM, or any range or value therebetween. In some embodiments, a compound of the invention is present in a composition at a concentration of 1-10 μM, 5-20 μM, 10-30 μM, 20-40 μM, 25-50 μM, 30-60 μM, 40-70 μM, 50-80 μM, 65-90 μM, 70-100 μM, 80-110 μM, 90-120 μM, 110-160 μM, 150-275 μM, 250-500 μM, 400-700 μM, 650-850 μM, or 800-1,000 μM within the composition. Each possibility represents a separate embodiment of the invention.

As defined herein, the term “half maximal inhibitory concentration (IC₅₀)” refers to a measure of the potency of a compound to inhibit a specific biological or biochemical function. In some embodiments, a compound of the invention or a composition comprising thereof has an IC₅₀ at the micromolar level. In some embodiments, micromolar level comprises 1,200 μM at most, 900 μM at most, 800 μM at most, 700 μM at most, 600 μM at most, 500 μM at most, 400 μM at most, 300 μM at most, 200 μM at most, 100 μM at most, 75 μM at most, 50 μM at most, 35 μM at most, 20 μM at most, 15 μM at most, 10 μM at most, 5 μM at most, 1 μM at most, 0.1 μM, 50 nM, or any range or value therebetween. In some embodiments, micromolar level comprises 5-100 nM, 50-500 nM, 400-1,200 nM, 1-10 μM, 5-20 μM, 15-30 μM, 25-500 μM, 40-75 μM, 70-120 μM, 100-200 μM, 150-300 μM, 250-400 μM, 375-500 μM, 400-650 μM, 600-850 μM, or 800-1,000 μM. Each possibility represents a separate embodiment of the invention.

As used herein, the term “carrier” refers to a diluent, adjuvant, excipient, or vehicle with which the active compound is administered. Such carriers can be sterile liquids, such as water-based and oils, including those of petroleum, animal, vegetable or synthetic origin, such as peanut oil, soybean oil, mineral oil, sesame oil and the like, polyethylene glycols, glycerin, propylene glycol or other synthetic solvents.

Water may be used as a carrier such as when the active compound is comprised by a pharmaceutical composition being administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from about 0.1% to about 99.99999% by weight of the compositions presented herein.

An embodiment of the invention relates to compounds of the present invention, presented in unit dosage form and prepared by any of the methods well known in the art of pharmacy. In one embodiment, the unit dosage form is in the form of a tablet, capsule, lozenge, wafer, patch, ampoule, vial or pre-filled syringe.

In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

In one embodiment, the composition of the present invention is administered in the form of a pharmaceutical composition comprising at least one of the active components of this invention together with a pharmaceutically acceptable carrier or diluent. In another embodiment, the composition of the invention can be administered either individually or together in any conventional oral, parenteral or transdermal dosage form.

As used herein, the terms “administering”, “administration”, and like terms refer to any method which, in sound medical practice, delivers a composition containing an active agent to a subject in such a manner as to provide a therapeutic effect.

In some embodiments, the pharmaceutical composition comprising the molecules of the invention, or combination thereof, are administered via oral (i.e., enteral), rectal, vaginal, topical, nasal, ophthalmic, transdermal, subcutaneous, intramuscular, intraperitoneal or intravenous routes of administration. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. In addition, it may be desirable to introduce the pharmaceutical composition of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

For topical application, the molecules of the invention, or combination thereof can be combined with a pharmaceutically acceptable carrier so that an effective dosage is delivered, based on the desired activity. The carrier can be in the form of, for example, and not by way of limitation, an ointment, cream, gel, paste, foam, aerosol, suppository, pad or gelled stick.

For oral applications, the pharmaceutical composition may be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. The tablets of the invention can further be film coated.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

The composition also includes incorporation of the active material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such composition will influence the physical state, solubility, stability, rate of in vivo release, and rate of in vivo clearance.

In one embodiment, the present invention provides combined preparations. In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In one embodiment, it will be appreciated that the molecules of the invention, or combination thereof, can be provided to the individual with additional active agents to achieve an improved therapeutic effect as compared to treatment with each agent by itself. In another embodiment, measures (e.g., dosing and selection of the complementary agent) are taken to adverse side effects which are associated with combination therapies.

In one embodiment, depending on the severity and responsiveness of the condition to be treated, dosing can be of a single or a plurality of administrations, with course of treatment lasting from several days to several weeks or until cure is affected or diminution of the disease state is achieved.

In some embodiments, the composition of the preset invention is administered in a therapeutically safe and effective amount. As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the presently described manner. In another embodiment, a therapeutically effective amount of the molecules, or any derivative or combination thereof, is the amount of the mentioned herein molecules necessary for the in vivo measurable expected biological effect. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005). In some embodiments, preparation of effective amount or dose can be estimated initially from in vitro assays. In one embodiment, a dose can be formulated in animal models and such information can be used to more accurately determine useful doses in humans.

In one embodiment, the compound or the salt thereof is administered at a daily dose in a range between 0.1 and 10,000 mg/kg/day, 0.1 and 1 mg/kg/day, 1 and 10 mg/kg/day, 10 and 20 mg/kg/day, 20 and 30 mg/kg/day, 20 and 100 mg/kg/day, 30 and 40 mg/kg/day, 40 and 50 mg/kg/day, 50 and 60 mg/kg/day, 60 and 70 mg/kg/day, 70 and 80 mg/kg/day, 80 and 90 mg/kg/day, 90 and 100 mg/kg/day, 100 and 150 mg/kg/day, 150 and 200 mg/kg/day, 200 and 300 mg/kg/day, 300 and 400 mg/kg/day, 400 and 500 mg/kg/day, 500 and 600 mg/kg/day, 700 and 800 mg/kg/day, 800 and 900 mg/kg/day, 900 and 1000 mg/kg/day, 1000 and 1500 mg/kg/day, 1500 and 2000 mg/kg/day, 2000 and 5000 mg/kg/day, 5000 and 6000 mg/kg/day, 6000 and 7000 mg/kg/day, 7000 and 8000 mg/kg/day, 8000 and 10,000 mg/kg/day, including any range or value therebetween.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p.1].

Pharmaceutical compositions containing the presently described molecules or combination thereof, as the active ingredient can be prepared according to conventional pharmaceutical compounding techniques. See, for example, Remington's Pharmaceutical Sciences, 18th Ed., Mack Publishing Co., Easton, Pa. (1990). See also, Remington: The Science and Practice of Pharmacy, 21st Ed., Lippincott Williams & Wilkins, Philadelphia, Pa. (2005).

In one embodiment, compositions including the preparation of the present invention formulated in a compatible pharmaceutical carrier are prepared, placed in an appropriate container, and labeled for treatment of an indicated condition.

In one embodiment, there is a kit comprising the compound of the invention or the salt thereof. In one embodiment, there is a kit comprising the composition of the invention.

In one embodiment, compositions of the present invention are presented in a pack or dispenser device, such as an FDA approved kit, which contains, one or more unit dosages forms containing the active ingredient. In one embodiment, the pack, for example, comprises metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

By another aspect, there is provided a method of inhibiting or reducing bacterial growth. In some embodiments, the method comprises contacting the bacteria with a composition comprising one or more compounds of the invention.

By another aspect, there is provided a method of treating or inhibiting a bacterial infectious disease or a symptom thereof in a subject in need thereof in some embodiments, the method comprises administering to the subject a therapeutically effective amount of a pharmaceutical composition comprising one or more compounds of the invention.

As used herein, “bacterial infectious disease” refers to any disease or disorder caused to a subject by an increased load of a microorganism. In some embodiments, the microorganism is bacteria. In some embodiments, the bacterial infectious disease is induced by or enhanced by Gram positive bacteria. In some embodiments, the bacterial infectious disease is induced by or enhanced by Gram negative bacteria. In some embodiments, the bacterial infectious disease is induced by a bacteria belonging to the genus Mycobacterium. In some embodiments, the bacterial infectious disease is induced by a bacteria belonging to the genus Staphylococcus.

Non-limiting examples of Staphylococcus species include but are not limited to: S. aureus, S. argenteus, S. schweitzeri, S. simiae, S. auricularis, S. carnosus, S. condimenti, S. debuckii, S. massiliensis, S. piscifermentans, S. simulans, S. capitis, S. caprae, S. epidermidis, S. saccharolyticus.

Non-limiting examples of Mycobacterium species include but are not limited to: M. tuberculosis, M. africanum, M. bovis, M. canetti, M. caprae, M. microti, M. mungi, M. orygis, M. pinnipedii, M. suricattae.

In some embodiments, a specie belonging to the genus Mycobacterium is Mycobacterium tuberculosis.

In some embodiments, a specie belonging to the genus Staphylococcus is Staphylococcus aureus.

In some embodiments, the bacterial infectious disease is induced or enhanced by a drug-resistant bacteria. In some embodiments, the drug-resistant bacteria is resistant to one or more types of drugs. In some embodiments, a drug as used herein is an antibiotic drug.

As used herein, one or more comprises at least 2, at least 3, at least 4, at least 5, at least 6, at least 7, at least 8, at least 9, at least 10 drugs, or any value or range therebetween. In some embodiments, one or more comprises 2 to 4, 2 to 7, 2 to 10, 3 to 8, 4 to 6, 4 to 9, 5 to 8, 5 to 10, 6 to 8, 7 to 9, 7 to 10, or 8 to 10 drugs. Each possibility represents a separate embodiment of the invention.

Non-limiting examples for symptoms of an infectious disease include, but are not limited to, fever, diarrhea, fatigue, muscle aches and coughing.

Non-limiting infectious diseases include urinary tract infection, gastrointestinal infection, enteritis, salmonellosis, diarrhea, tuberculosis, nontuberculous mycobacterial infections, legionnaires' disease, hospital-acquired pneumonia, skin infection, cholera, septic shock, periodontitis, infection, and sinusitis. In some embodiments, the infection induces a condition selected from the group consisting of: bacteremia, skin infections, neonatal infections, pneumonia, endocarditis, osteomyelitis, toxic shock syndrome, scalded skin syndrome, and food poisoning.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, and pig. In one embodiment, the subject is a human. Human subjects may also include fetuses.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described peptides prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, the term “condition” includes anatomic and physiological deviations from the normal that constitute an impairment of the normal state of the living animal or one of its parts, that interrupts or modifies the performance of the bodily functions.

In some embodiments, the composition of the present invention is directed to killing microorganisms in a living tissue or on or in an article or reducing the formation of microorganisms on or in an article.

In some embodiments, there is provided a use of a composition comprising an effective amount of the compound of the invention in the preparation of a medicament for the treatment, amelioration, reduction, or prevention of a bacterial infectious disease or a symptom thereof in a subject in need thereof. In some embodiments, the invention provides use of a composition comprising an effective amount of one or more of the molecules of the invention or a combination thereof in the preparation of a medicament for the treatment of a bacterial infectious disease or a symptom thereof in a subject in need thereof.

In one embodiment, the compound of the invention is provided to the subject per se. In one embodiment, one or more of the compounds of the invention are provided to the subject per se. In one embodiment, the compound of the invention is provided to the subject as part of a pharmaceutical composition where it is mixed with a pharmaceutically acceptable carrier. In one embodiment, one or more of the compounds of the invention are provided to the subject as part of a pharmaceutical composition where they are mixed with a pharmaceutically acceptable carrier.

As used herein, the term “about” when combined with a value refers to plus and minus 10% of the reference value. For example, a length of about 1,000 nanometers (nm) refers to a length of 1,000±100 nm.

It is noted that as used herein and in the appended claims, the singular forms “a”, “an”, and “the” include plural referents unless the context clearly dictates otherwise. Thus, for example, reference to “a compound” includes a plurality of such compounds and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

### Fragment Based Virtual Screening

According to some aspects of the present invention, there is provided a method for identifying a compound that binds to a biological target. In some embodiments, there is a method of screening compounds for determining a drug candidate. In some embodiments, the biological target is an enzyme. In some embodiments, the biological target is a ribosomal PTC. In some embodiments, the biological target is PTC of S. aureus. In some embodiments, the enzyme is associated with DNA or RNA processing. In some embodiments, the target is a polynucleotide (such as DNA or RNA).

In some embodiments, DNA or RNA processing is selected from replication, and translation. In some embodiments, the enzyme is selected from the group consisting of: ribosome, RNAzyme, DNAzyme, DNA-Gyrase, polymerase, primase, and helicase.

In some embodiments, the biological target is selected from poly-RNA and poly-DNA molecules.

According to another aspect, the method comprises performing T₂ relaxation NMR on a sample comprising a target and a plurality of fragment molecules, thereby determining a relaxation time of the fragment molecules. In some embodiments, the method comprises evaluating T2 relaxation time of a fragment molecule by performing T₂ relaxation NMR. In some embodiments, the method comprises evaluating T2 relaxation time of a fragment molecule by performing T₂ relaxation NMR, thereby determining a lead fragment having a binding affinity to the biological target. In some embodiments, the method is for evaluating a lead fragment characterized by a highest binding affinity to the biological target.

In some embodiments, the sample comprises 1 to 50 fragment molecules and D₂O as a solvent. In some embodiments, the sample comprises the biological target (such as a target polynucleotide sequence), the fragment molecule and a deuterated solvent (such as D₂O). In some embodiments, the molar ratio of the fragment molecule relative to the biological target is in the range from 2 to 200, from 2 to 10, from 10 to 20, from 20 to 40, from 40 to 60, from 60 to 80, from 80 to 100, from 100 to 150, from 150 to 200 including any range or value therebetween.

In some embodiments, the target polynucleotide sequence comprises poly-RNA. In some embodiments, the target sequence comprises poly-DNA.

In some embodiments, the NMR scan is performed on a sample comprising fragment molecules. In some embodiments, the NMR scan is performed on a sample comprising fragment molecules and the target sequence.

In some embodiments, the pulse sequence is a Carr-Purcell-Meiboom-Gill (CPMG) sequence. In some embodiments, the pulse sequence is a differential line broadening (DLB) sequence.

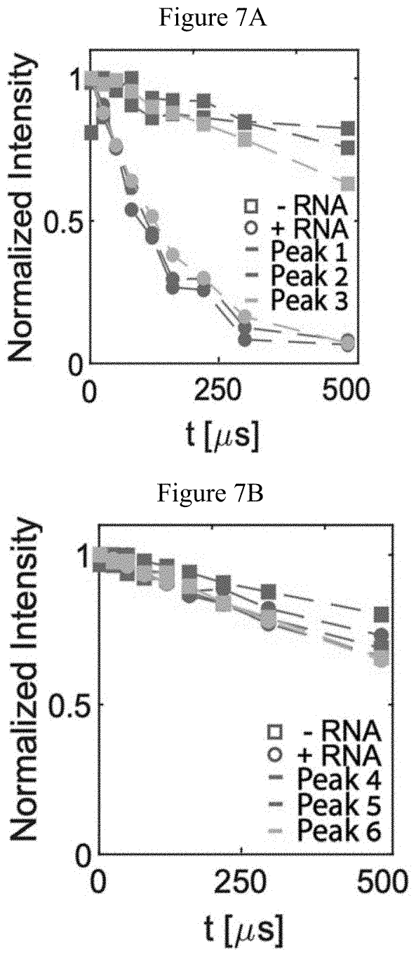

In some embodiments, the method comprises performing a T₂ relaxation NMR scan of a sample, comprising applying a pulse sequence to the sample, recording free induction decay (FID) data and analyzing the recorded FID data to obtain a 1D CPMG or differential line broadening (DLB) spectrum of the sample. In some embodiments, the method comprises analyzing the recorded FID data to calculate the T2 relaxation time, thereby estimating binding affinity of the fragment molecule to the biological target. An exemplary T2 analysis based on 1D CPMG spectra is represented by FIGS. 7A-B.

The main principle of relaxation NMR is that a fragment molecule will exhibit slower relaxation upon binding to a large target sequence. Thus, the binding is detected by measuring a relaxation time of the fragment molecule. In some embodiments, the fragment molecule having a binding affinity to the target sequence is characterized by a greater T2 relaxation time. In some embodiments, binding affinity is estimated by comparing H¹NMR signal intensity of the fragment molecule with and without the target sequence. In some embodiments, H¹NMR signal intensity relates to one or more protons of the fragment molecule.

In some embodiments, analyzing FID data comprises calculating a relaxation time of a fragment molecule. In some embodiments, the relaxation time is calculated by analyzing a DLB for the line width at half height of a peak. In some embodiments, the relaxation time is calculated by determining a magnetization decay rate (R₂).

In some embodiments, a binding of a fragment molecule to the target sequence is confirmed by comparing the relaxation time of the free fragment molecule to the relaxation time of the fragment molecule in a complex with the target sequence.

In some embodiments, fragment molecules or lead fragments that bind to the target sequence are ranked for their binding affinity. In some embodiments, ranking is based on the best relaxation time value (such as greater relaxation time). In some embodiments, the lead fragment comprises the fragment molecule with the highest binding affinity to the biological target.

In some embodiments, a fragment molecule having a binding affinity to the biological target is characterized by at least 10%, at least 20%, at least 30%, at least 40%, at least 50%, at least 60%, at least 70%, at least 80%, at least 90%, greater relaxation time as compared to a fragment molecule being devoid of binding affinity to the biological target, wherein the relaxation time is measured by T₂ relaxation NMR. In some embodiments, relaxation time of the lead fragment is at least 50%, at least 60%, at least 70%, at least 80%, at least 90% of the relaxation time of the biological target, as measured by T₂ relaxation NMR.

According to another aspect, the method further comprises generating a dataset comprising a plurality of virtual molecules, wherein the virtual molecule comprises a fragment having at least 70% homology to the lead fragment. In some embodiments, the virtual molecule comprises a fragment having at least 75%, at least 80%, at least 85%, at least 90%, at least 95%, at least 99%, homology to the lead fragment. In some embodiments, the homology is similarity or identity. In some embodiments, the homology is structural homology. In some embodiments, the virtual molecule comprises a fragment identical to the lead fragment.

In some embodiments, the method comprises optimization of the lead fragment by generating the dataset comprising a plurality of virtual molecules. In some embodiments, generating a dataset is by performing virtual filtration. In some embodiments, the optimization of the lead fragment comprises a step of virtual filtration and a step of molecular docking.

In some embodiments, the optimization comprises generating the dataset by virtual filtration, and estimating values of binding energy (ΔG) for at least a part of the plurality of virtual molecules. In some embodiments, binding energy is related to binding affinity of the virtual molecule to the biological target. In some embodiments, the dataset comprises a plurality of virtual molecules and corresponding ΔG values. In some embodiments, the dataset is received by virtual filtration and high throughput docking.

In some embodiments, virtual filtration comprises selecting molecules from a dataset based on their structural similarity to the fragment molecule selected by NMR based screening. In some embodiments, the similarity is defined as at least 70% of structural identity. In some embodiments, the dataset comprises a virtual small molecules library (such as ZINC library).

In some embodiments, the optimization further comprises performing a docking of the virtual molecules from the dataset against a structure of the target, thereby receiving corresponding ΔG values.

In some embodiments, the method comprises performing high throughput docking on the dataset to obtain values of ΔG for any of the virtual molecules, thereby identifying a compound capable of binding to the biological target. In some embodiments, the compound capable of binding to the biological target has the lowest ΔG value. In some embodiments, the compound having greater affinity to the biological target has lowest ΔG value.

After performing the virtual filtration, a virtual library may be generated containing drug-like compounds. Molecular docking may be performed to estimate the binding free energy of the drug-like compounds to the receptor. By screening the virtual library, compounds are ranked based on their ΔG value. Compounds with highest binding value (i.e. lowest ΔG value) were selected for the next optimization step.

In some embodiments, the method further comprises performing an in-vitro test to evaluate binding properties of the compound from the virtual dataset to the biological target. In some embodiments, the compound is tested by performing in-vitro translation assay, as described hereinbelow (Examples section).

According to another aspect, there is a method for predicting a binding strength of molecules to the target by applying Machine Learning. In some embodiments, the method is for predicting corresponding ΔG values of molecules from the virtual dataset. In some embodiments, the virtual dataset is as described hereinabove.

In some embodiments, there is a system and/or a method for predicting binding energy values of molecules to a biological target. In some embodiments, the system and/or the method takes into account structure-activity based parameters of each molecule.

In some embodiments, the system comprises at least one hardware processor; and a non-transitory computer-readable storage medium having stored thereon program instructions, the program instructions executable by the at least one hardware processor to: receive, as input, a dataset comprising a plurality of virtual molecules and corresponding values of binding energy (ΔG) with respect to a biological target; calculating a plurality of features for each of the virtual molecules; at a training stage, train a Machine Learning (ML) model on a training set comprising: a. the plurality of features, and b. labels associated with the corresponding values of ΔG; and at an inference stage, apply the ML model to a new virtual molecule to predict a ΔG value with respect to the biological target.

In some embodiments, the biological target is as described hereinabove.

In some embodiments, the system receives, as input, a dataset comprising a plurality of virtual molecules and corresponding ΔG values from virtual filtration and docking stages, as described hereinabove. In some embodiments, the dataset comprises molecules based on the same lead fragment. In some embodiments, the lead fragment corresponds to at least a part of the virtual molecule, wherein the lead fragment is identified by T2 relaxation NMR as described hereinabove.

In some embodiments, the dataset comprises virtual molecules based on a similar lead fragment. In some embodiments, the dataset comprises coordinates of each atom which belongs to the lead fragment of the virtual molecule. In some embodiments, the lead fragment is as described hereinabove.

In some embodiments, the plurality of features comprises a variable selected form the group consisting of: DIST, MOD and VAR or any combination thereof. In some embodiments, the variable is independent variable. In some embodiments, the variable is associated with coordinates of each atom belonging to the lead fragment. In some embodiments, each atom belonging to the lead fragment has a plurality of variables associated therewith.

In some embodiments, the variable MOD defines whether a substituent is bound to the atom. In some embodiments, the variable MOD comprises true and false defining whether a substituent is bound to a particular atom position. In some embodiments, the variable DIST represents a distance between a pair of atoms. In some embodiments, the variable DIST represents a distance between a pair of atomic coordinates. In some embodiments, the variable VAR represents the variance of DIST. In some embodiments, the variable VAR is an average distance within the virtual molecule. In some embodiments, the variable VAR corresponds to a volume of the virtual molecule.

In some embodiments, the dataset further comprises the label associated with the ΔG values corresponding to each molecule. In some embodiments, the label associated with the ΔG values is referred to as BOND.

In some embodiments, ML model is applied to select from a dataset a molecule characterized by lowest value of ΔG. In some embodiments, ML model is applied to sort molecules within the dataset according to the corresponding ΔG values. In some embodiments, ML model is applied to predict a feature significant for lowering the ΔG value of the virtual molecule. In some embodiments, ML model is applied to predict the lowest ΔG value of the virtual molecule with respect to one or more features.

As extensively demonstrated in the Examples section the ML model is applied to evaluate the most significant features for lowering the ΔG value of the virtual molecule.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

### Materials and Methods

Chemicals were from Sigma. Creatine phosphate and creatine kinase were from Roche Molecular Biochemicals. Luciferase assay kit was purchased from Promega. Small molecule inhibitors were purchased from UORSY, Ukraine. RNase T1 and cAMP were from Thermo Scientific. Quick calf intestinal alkaline phosphatase and T4 polynucleotide kinase were from New England Biolabs. [g-³²P]-ATP (800 Ci mmol⁻¹) were from Perkin Elmer.

RNA preparation

29 mer RNA, SEQ ID NO: 1 (GCAUCCUGGGGCUGGAGCAGGUCCCAAGG) representing the sequence of 23S rRNA of Mtb (2543-2571) for NMR screening was purchased.

The full-length rRNA of Mtb (GeneBank: NC_000962.3, 1473658 . . . 1476795) was cloned in a pLitmus28 vector, and small fragments of the PTC were obtained. RNA in vitro transcription was carried out in a reaction mixture containing 8 g linearized plasmid, 18.5 μM T7 RNA polymerase, 1 mM ATP, CTP, GTP and UTP, 10 units RNase inhibitor, in appropriate reaction buffer (80 mM HEPES-KOH pH 7.5, 10 mM Spermidine, 40 mM DTT, 25 mM MgCl₂). Reactions (100 μL) were incubated at 37° C. for 4 hours.

T2-Relaxation Experiment

10 mM of RNA in a sample volume of 170 uL was used in the initial screening. In the initial screening 10 fragment molecules from The Maybridge Ro3 2500 Diversity Fragment Library (Maybridge, Inc.) were mixed with 29mer RNA molecule SEQ ID NO: 1 that represents hairpin 91 of M. tuberculosis PTC (2543-2571) and exposed to NMR (T2 relaxation) screening. As the samples were arranged in pools of 10 compounds and mixed with 10 mM RNA (total of 20 ml). For a ligand detected screen the RNA had to be in a proton free phosphate buffer (PBS). The RNA sample was measured for stability using denaturing gel analysis (i.e. not aggregated or unfolded).

Fragment Based Drug Discovery by T2 Relaxation

The Carr Purcell Maiboom Gill (CPMG) sequence was used to determine the relaxation time T2 of the small molecules in their free form and in the presence of the biomolecule. The relaxation times are indirectly proportional to the line width at half height. The large difference in relaxation times of the bound and unbound form can also be used to filter out resonances originating from the biomolecule and the complex, for example, by using a long echo time in the CPMG experiment, or by adding a filter to the STD experiment. A ligand bound to a larger biomolecule diffuses at a much slower pace than the free ligand. The diffusion rate of the free ligands as well as the diffusion rate of ligand in the presence of target biomolecule can be measured by Diffusion Ordered Spectroscopy NMR (DOSY-NMR).

In Silico Molecular Docking

Molecular docking simulations were performed using AutoDock 4.2 to estimate the binding free energy (ΔG) and the poses of the investigated compounds regarding the receptor. The PTC receptor for simulations was derived from the Cartesian coordinates of the large ribosomal subunit of SA50S originated from Staphylococcus aureus (PDBID 4WCE). Cognate ligands chosen for this purpose were imported from the ZINC database. Virtual screening protocol was conducted through the Raccoon implementation, by which the receptor and ligand molecules were preprocessed for docking. A docking grid was set with 126 points in each dimension and a default spacing of 0.375 Å. The received grid map was centered with respect to the receptor. Free energy calculations and conformational sampling of the ligands were then carried out through Lamarckian genetic algorithm (LGA), with an initial population size of 150 individuals, 2,500,000 free energy evaluations and 27,000 LGA generations. Clustering of the results was performed via root mean-square deviation (RMSD) calculations for the obtained poses of the ligands, with a constant tolerance of 2.0 Å. Further analyses of the results were performed using default AutoDock VS tutorial scripts along with several in-house scripts.

A Machine Learning (ML) Approach to Inhibitors' Design

### Data Preparation

The data contained 811 molecules with a common scaffold. The remaining 107 molecules from the initial number of molecules used (919) were aligned in another coordinate system, and therefore were removed. Each molecule was composed of 20 to 51 atoms, 33 on average, 11 of which belong to the scaffold. The raw data contained the coordinates of each atom relative to the center of the molecule. The coordinates were used to compute a total of 33 features per molecule. The atoms of the scaffold were numbered from 1 to 11 and for every location i three features were considered (33 overall). The feature DISTi is a proxy to the volume of the structure connected at location i—the larger the average distance, the bigger the structure. The feature VARi is a proxy to the measure of regularity or smoothness of the structure—the smaller the variance the more regular the structure. In addition to the 33 independent variables, the dependent variable for each molecule was BOND, which holds the binding energy given by the docking results.
At least one of locations 1, 2, 4, 5, 6, 8, and 9 was modified in 98% to 100% of the molecules. On the other hand, locations 3, 7 and 10 were never modified. Location 11 was modified in 1% of molecules. The binding energy BOND ranged from −15.680 (strongest) to −4.77 (weakest). The average was −9.85 with standard deviation of 1.89.

### ML Algorithms Used:

a. Random forest. Random forests is a well-known and widely used ensemble learning method for classification or regression that operates by constructing a multitude of regression trees at training time and outputting a value that is the average of the predictions of the individual trees. Random forests are widely used in a multitude of ML tasks and are considered to be among the top methods in terms of predictive power. The main advantage of Random Forests over linear regression is the fact that the regression surface is not linear. This allows to provide better predictions. In each regression tree, the data is split recursively into nodes. Each node is split into two children nodes according to a carefully selected if-then question about a certain feature. For example, if x.DIST8>1.75 then place molecule x in the left son, otherwise in the right son. The criterion for choosing the question is the minimization of pooled variance after the split. When certain predefined stopping conditions are met, the node is no longer split and turns to a leaf, whose value is the average value of items in that node (in our case, the average binding energy of the molecules in that leaf). In the prediction phase, a molecule is run through the tree, branching left or right according to the answer to the specific question at each node. When reaching a leaf, the value of that leaf is returned as the prediction value.

At the training stage a random forest with 50 trees was used, with no restriction on the tree-depth or the number of features to be considered for each node split. The RMSE (root mean squared error) of the model prediction was 0.56 (less than third of the standard deviation of BOND). A second random forest model was trained, using only the 11 MODi features. The resulting RMSE was 1.86, more than three times larger. Alongside the RMSE, a useful and commonly used statistic of the random forest is called feature importance. The feature importance is computed as follows: for every feature, all the splits where this feature was used as the splitting criterion are examined, and the average decrease in variance is computed (variance of the node before the split minus the pooled variance of the two children nodes after the split). The larger the difference the more important is the feature. The features were sorted according to their feature importance value: location 8 is the most important one, then—by an order of magnitude—locations 6, 9 and 5. The remaining locations were at least another order of magnitude lower.

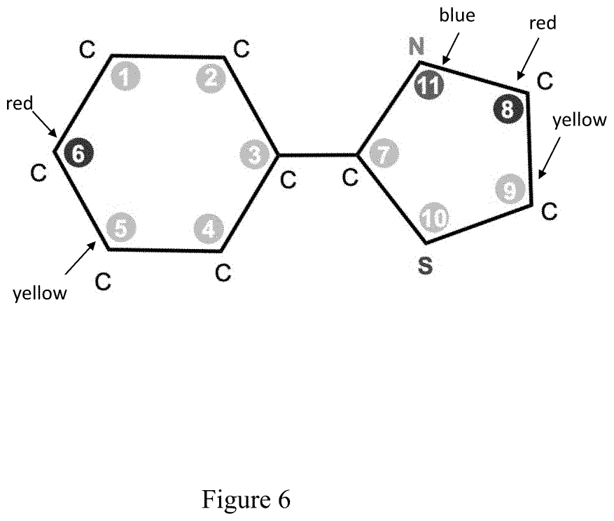

b. Linear Regression. A regression model was trained and used the step function to select significant features. The significant features (with p-value smaller than 0.05) were (in order of significance) DIST8, MOD8, VAR8, DIST5, DIST9 and VAR6. We then regressed BOND on those features and obtained a regression model with Adjusted R² value of 0.23, F(6,804) was 41.96 and p-value: <2.2e⁻¹⁶.
The fact that the coefficient of all the DIST variables is negative implies that increasing their value will decrease (strengthen) BOND. Similarly, the fact that VAR8 is positive—increasing VAR8 increases (weakens) BOND. Next, we regressed BOND against the six variables in the above table, and in addition all the pairwise interaction variable (e.g. VAR6*DIST5). The regression Adjusted R² increased to 0.25 and F(15,795)=19.11 giving p-value below 2.2e-16. Interestingly, VAR6 became insignificant, and rather the product VAR6*DIST8 became significant. The second significant interaction was VAR8*DIST8 with a negative coefficient −0.9. VAR8 and DIST8 remained significant with coefficients 5.88 and −4.25 respectively. This means that to have a strong bonding energy, i.e. negative BOND, the structure attached at location 8 needs to be either very regular and large, or irregular and large so that the product −4.25*VAR8*DIST8 is much larger than 5.88*VAR8-4.25*DIST8. This is feasible since the product grows faster than the linear terms. Structure-activity-relationship (SAR) of a virtual phenyl-thiazol based PTC inhibitor, as predicted by ML model is represented in FIG. 6. Based on the ML model described herein, it was possible to identify 10 potential PTC inhibitors (Table 1). Some of these molecules exhibited a superior PTC inhibition at low micromolar range, as exemplified hereinbelow.

### Example 1

### Mycobacterial PTC Inhibitors

The inhibition effect of compounds (entry Nrs. 1-10, listed in Table 1) and chloramphenicol on M. smegmatis ribosomes was tested in a bacterial coupled transcription/translation assay, where the expression of luciferase gene was measured. The luciferase gene was inserted into a plasmid downstream from the T7 RNA polymerase promotor. The reaction mixture contained: 160 mM HEPES-KOH (pH 7.5), 6.5% polyethylene glycol 8 000, 0.074 mg/ml tyrosine, 1.3 mM adenosine triphosphate (ATP), 0.86 mM cytidine triphosphate (CTP), guanosine triphosphate (GTP) and uridine triphosphate (UTP), 208 mM potassium glutamate, 83 mM creatine phosphate, 28 mM ammonium acetate, 0.663 mM cyclic adenosine monophosphate (cAMP), 1.8 mM dithiothreitol (DTT), 0.036 mg/ml folinic acid, 0.174 mg/ml E. coli tRNA mix, 1 mM amino acid, 0.25 mg/ml creatine kinase, 0.044 mg/ml T7 RNA polymerase, E. coli cell-free extract, 0.003 g/l luciferase-encoding plasmid and compounds Nr. 1-10 in concentration ranging from 152 nM to 1 mM. The effect of these compounds, at a final concentration of 300 nM, was also tested against M. smegmatis ribosomes. The reaction mixture was incubated at 37-C for 1 h and terminated by the addition of erythromycin at a final concentration of 8 μM. To quantify the reaction products, luciferin assay reagent (LAR, Promega) was added at 5:3 (luciferase: reaction mix) volume ratio and luminescence was measured. The results were plotted (compound concentration vs. luminescence intensity) and IC₅₀ values were calculated.

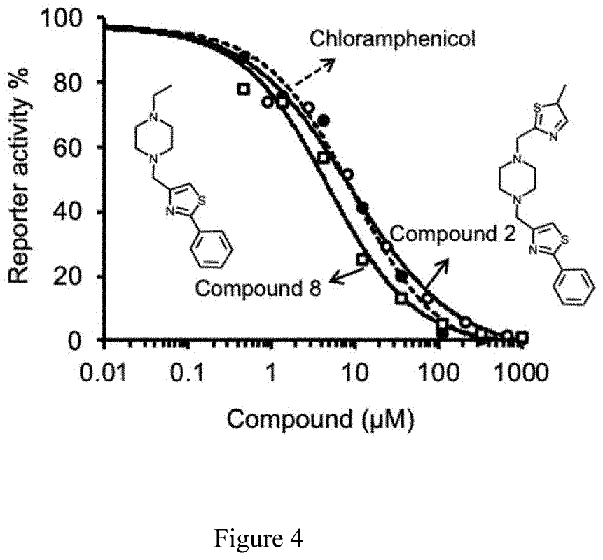

A group of phenylthiazole derivatives represented by Table 1 was predicted by ML model as effective Mtb PTC inhibitors. Compounds 2 and 8 exhibited in vitro potency against Mtb PTC as confirmed by their IC50 values, which were significantly improved over standard antibiotic drugs. Compounds 2 and 8 were shown to inhibit translation of a reporter gene by Mtb ribosomes in vitro (FIG. 4), comparably and even superiorly to common antibiotics (e.g., chloramphenicol, FIG. 4). The reduction of protein synthesis was reflected by reduced luciferase amounts (i.e., reduced fluorescence), which inversely correlated with the PTC inhibitor, in a dose-dependent manner. Compound Nr. 2 was shown to have higher inhibition activity (data not shown), compared to compounds Nr: 3, 4 and 5 (with the last two showing comparable inhibition capabilities).

Contrary to the above, compounds sharing structural similarity with the disclosed Mtb PTC inhibitors, having the following structural formulae:

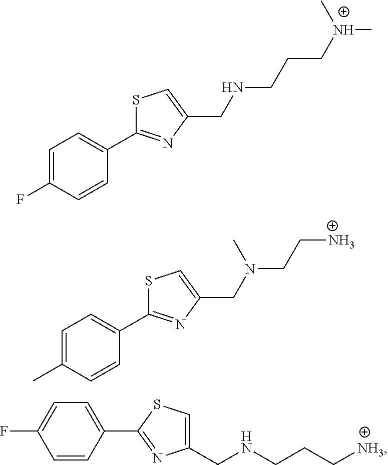

wherein Me is a methyl group, did not show inhibition of protein synthesis under the conditions of a Luciferase reporter assay, as used herein.

TABLE 1
representing 10 candidate molecules with the best
docking binding free energy with respect to PTC.

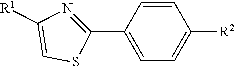

| Entry | R¹ | R² |
|---|---|---|
| 1 | 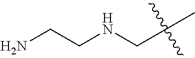 [SMILES: *CNCCN] | F |
| 2 | 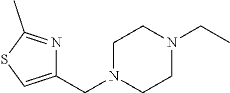 | H |
| 3 | 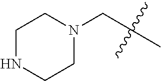 [SMILES: *CN1CCNCC1] | Me |
| 4 | 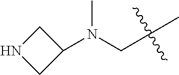 [SMILES: *CN(C)C1CNC1] | H |
| 5 | 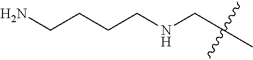 [SMILES: *CNCCCCN] | F |
| 6 | 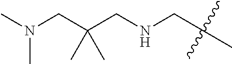 [SMILES: *CNCC(C)(C)CN(C)C] | Me |
| 7 | 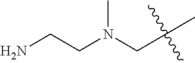 [SMILES: *CN(C)CCN] | Me |
| 8 | 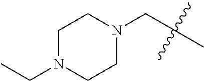 [SMILES: *CN1CCN(CC)CC1] | H |
| 9 | 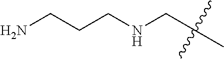 [SMILES: *CNCCCN] | F |
| 10 | 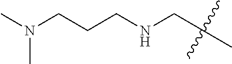 [SMILES: *CNCCCN(C)C] | F |

An additional set of potential PTC inhibitors (Table 2) has been synthesized, based on lead molecules of Table 1. The set of molecules listed in Table 2 undergo biological studies so as to evaluate the inhibitory potential of these molecules with respect to bacterial PTC (Mycobacterial and/or Staphylococcal PTC). The IC₅₀ values of these molecules will be determined.

TABLE 2
representing an additional set of candidate molecules.

| Entry | Structure |
|---|---|
| 11 | 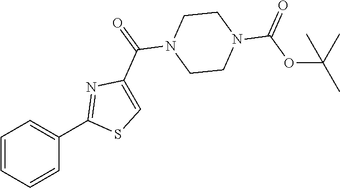 [SMILES: CC(C)(C)OC(=O)N1CCN(C(=O)c2csc(-c3ccccc3)n2)CC1] |
| 12 | 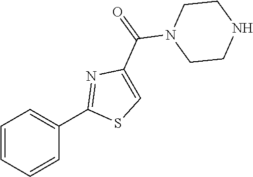 [SMILES: O=C(c1csc(-c2ccccc2)n1)N1CCNCC1] |
| 13 | 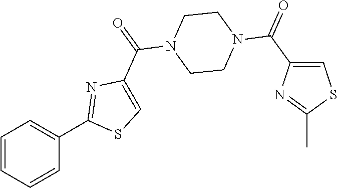 [SMILES: Cc1nc(C(=O)N2CCN(C(=O)c3csc(-c4ccccc4)n3)CC2)cs1] |
| 14 | 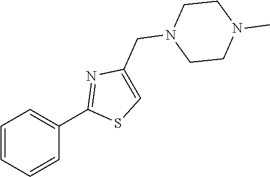 [SMILES: CN1CCN(Cc2csc(-c3ccccc3)n2)CC1] |
| 15 | 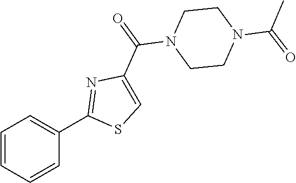 [SMILES: CC(=O)N1CCN(C(=O)c2csc(-c3ccccc3)n2)CC1] |
| 16 | 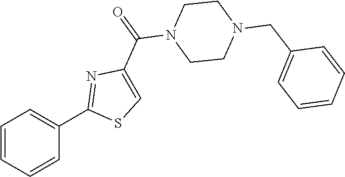 [SMILES: O=C(c1csc(-c2ccccc2)n1)N1CCN(Cc2ccccc2)CC1] |
| 17 | 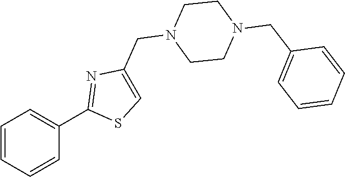 [SMILES: c1ccc(CN2CCN(Cc3csc(-c4ccccc4)n3)CC2)cc1] |
| 18 | 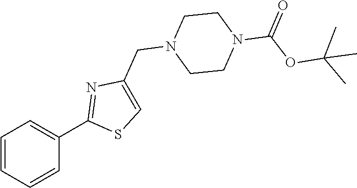 [SMILES: CC(C)(C)OC(=O)N1CCN(Cc2csc(-c3ccccc3)n2)CC1] |
| 19 | 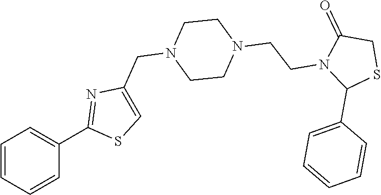 [SMILES: O=C1CSC(c2ccccc2)N1CCN1CCN(Cc2csc(-c3ccccc3)n2)CC1] |
| 20 | 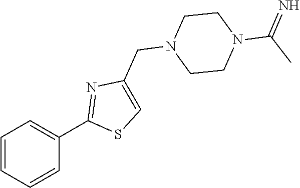 [SMILES: CC(=N)N1CCN(Cc2csc(-c3ccccc3)n2)CC1] |
| 21 | 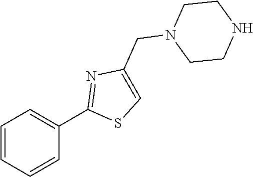 [SMILES: c1ccc(-c2nc(CN3CCNCC3)cs2)cc1] |
| 22 | 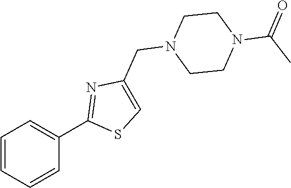 [SMILES: CC(=O)N1CCN(Cc2csc(-c3ccccc3)n2)CC1] |
| 23 | 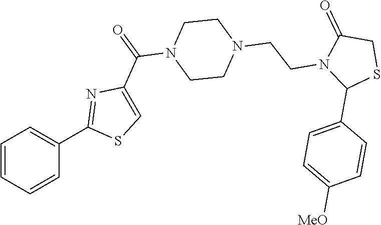 [SMILES: COc1ccc(C2SCC(=O)N2CCN2CCN(C(=O)c3csc(-c4ccccc4)n3)CC2)cc1] |
| 24 | 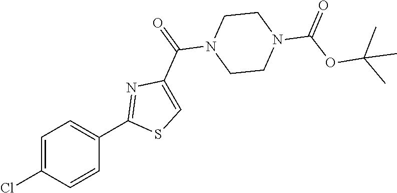 [SMILES: CC(C)(C)OC(=O)N1CCN(C(=O)c2csc(-c3ccc(Cl)cc3)n2)CC1] |
| 25 | 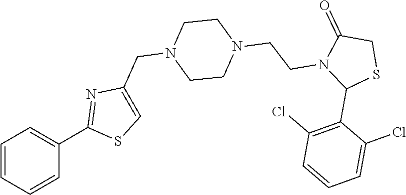 [SMILES: O=C1CSC(c2c(Cl)cccc2Cl)N1CCN1CCN(Cc2csc(-c3ccccc3)n2)CC1] |
| 26 | 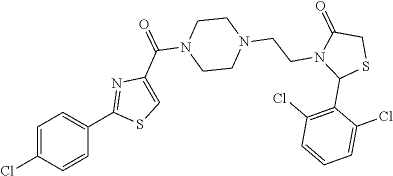 [SMILES: O=C(c1csc(-c2ccc(Cl)cc2)n1)N1CCN(CCN2C(=O)CSC2c2c(Cl)cccc2Cl)CC1] |
| 27 | 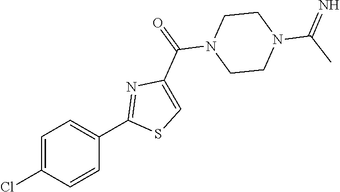 [SMILES: CC(=N)N1CCN(C(=O)c2csc(-c3ccc(Cl)cc3)n2)CC1] |
| 28 | 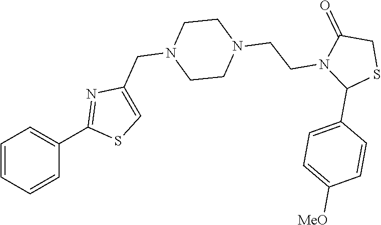 [SMILES: COc1ccc(C2SCC(=O)N2CCN2CCN(Cc3csc(-c4ccccc4)n3)CC2)cc1] |

These results show that the compounds disclosed herein inhibit pathogenic bacterial ribosomes, and in turn bacterial protein synthesis. Moreover, the disclosed Mtb PTC inhibitors were found to inhibit bacterial growth in vitro.

Accordingly, the compounds disclosed herein may be used as potent antimicrobial drugs, including but not limited to inhibiting Mycobacterium tuberculosis. Further, these compounds may be incorporated on or within articles, to thereby reduce the bacterial growth on the articles.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound or a salt thereof, said compound is represented by general Formula A: 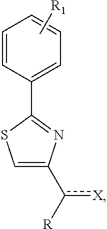
wherein:
R is selected from the group consisting of: 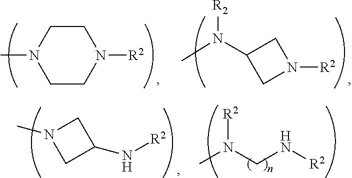
each R² independently comprises: a hydrogen atom, a linear or branched, and unsubstituted or substituted C₁-C₁₂ alkyl, aralkyl, aryl, carboxy group, imine, heteroaryl group, benzyl, a heterocyclyl, an alkyloxycarbonyl, an alkoxy group or any combination thereof;
X comprises an atom selected from the group consisting of: hydrogen, carbon, oxygen, nitrogen, and sulfur or any combination thereof; [symbol]
represents a double bond or a single bond;
n represents an integer being in a range between 1 and 40;
R¹ represents a substituent selected from the group consisting of a halogen atom, hydrogen, a nitro group, an amine group, an azide group, a cyano group, a methoxy group, a carboxylic group, an ester group, an ether group, an aromatic group, alkyl group, vinyl group, an alcohol, a carbamate group, a thiazole group, an urea group, and an alkyl group, or any combination thereof.

2. The compound of claim 1, wherein said compound is represented by general Formula I: 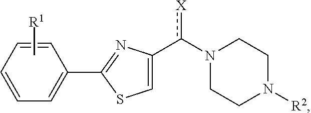
or by general Formula Ia:

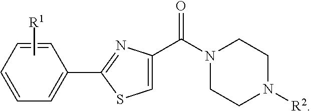

3. The compound of claim 1, wherein said X comprises an atom selected from the group consisting of: carbon, nitrogen, sulfur and oxygen, or any combination thereof, and wherein [symbol] represents a double bond.

4. The compound of claim 2, wherein each R² independently comprises: a linear or branched, and unsubstituted or substituted C₁-C₁₂ alkyl, aralkyl, aryl, carboxy group, imine, heteroaryl group, benzyl, a heterocyclyl, an alkyloxycarbonyl, an alkoxy group or any combination thereof; and wherein R¹ represents a substituent selected from the group consisting of: a halogen atom, an alkyloxycarbonyl, a hydrogen atom, an alkoxy group, and a C₁-C₅ alkyl group, or any combination thereof.

5. The compound of claim 1, wherein said compound comprises at least one of:

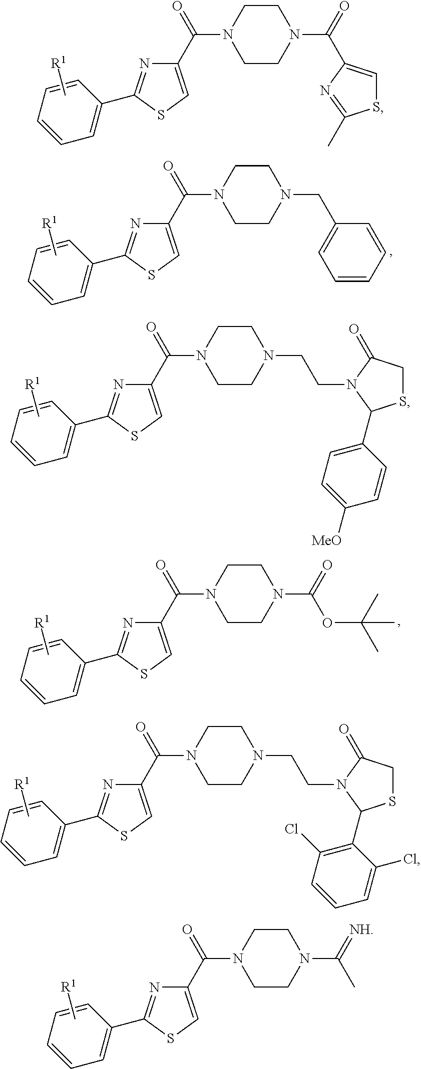

6. (canceled)

7. (canceled)

8. The compound of claim 1, wherein said compound comprises at least one of:

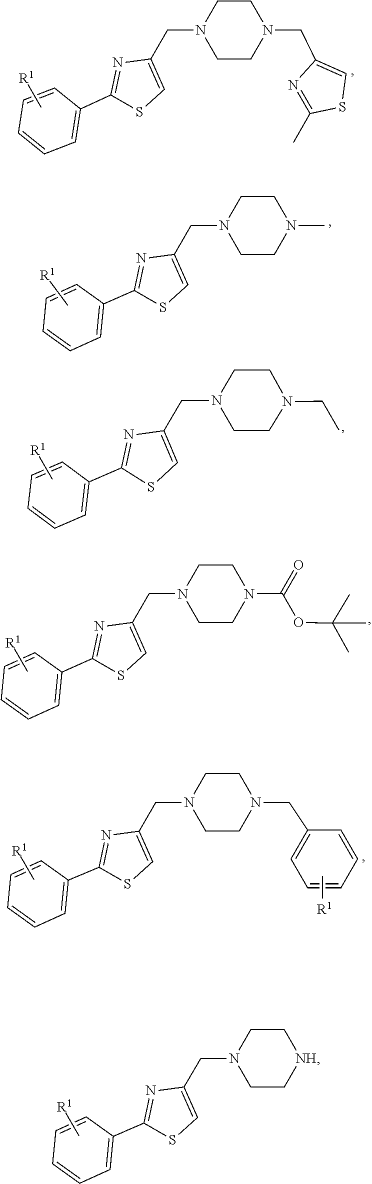

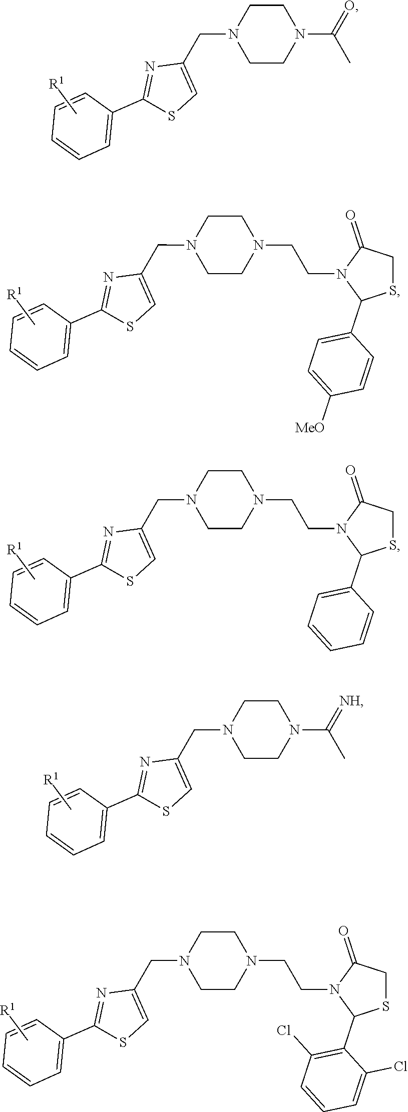

9. The compound of claim 1, represented by general Formula II: 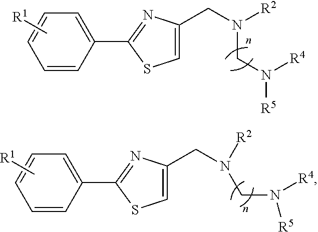
wherein:
R² is selected from a hydrogen atom, an alkyl, or is absent;
R⁴ and R⁵ are each independently selected from the group consisting of: a hydrogen atom, a straight or branched and unsubstituted or substituted C₁-C₁₂ alkyl, aralkyl, aryl, heteroaryl group, and any combination thereof; and
n is the number of carbons of a straight or branched and unsubstituted or substituted carbon chain ranging from 1 to 10.

10. The compound of claim 9, represented by general Formula IIa: 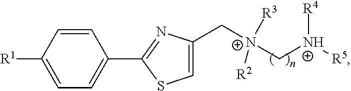
wherein each of R² and R³ is independently selected from a hydrogen atom, an alkyl, or is absent.

11. The compound of claim 9, wherein said R¹ represents a substituent selected from the group consisting of: a halogen atom (such as fluorine, chlorine, bromine or iodine), an alkyloxycarbonyl (such as tert-butyl-, ethyl-, butyl-, propyl-, iso-propyl-, and methyl-oxycarbonyl), a hydrogen atom, an alkoxy group (such as methoxy, an ethoxy, tert-butoxy), and a C₁-C₅ alkyl group, or any combination thereof, and wherein n is from 1 to 5.

12. The compound of claim 1, wherein said compound is represented by general Formula III: 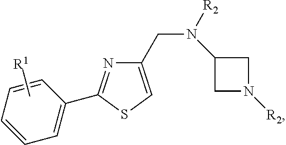
or by general Formula IIIa: 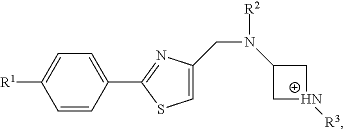
or by general Formula IV:

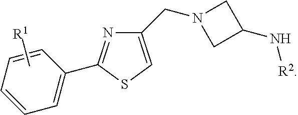

13. The compound of claim 12,
wherein said R¹ represents a substituent selected from the group consisting of: a halogen atom, an alkyloxycarbonyl, a hydrogen atom, an alkoxy group, and a C₁-C₅ alkyl group, or any combination thereof;
and wherein each of R² and R³ is independently selected from a hydrogen atom, an alkyl, or is absent.

14. (canceled)

15. The compound of claim 12, wherein said compound comprises: 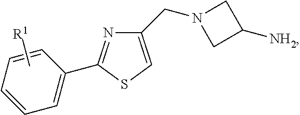
wherein R¹ represents a substituent selected from the group consisting of: a halogen atom, an alkyloxycarbonyl, a hydrogen atom, an alkoxy group, and a C₁-C₅ alkyl group, or any combination thereof.

16. An antibacterial composition comprising the compound of claim 1 or a salt thereof, and a pharmaceutically acceptable carrier.

17. The compound of claim 16, comprising an effective amount of said compound or said salt thereof.

18. (canceled)

19. (canceled)

20. (canceled)

21. A method for treating a bacterial infectious disease or a symptom thereof in a subject in need thereof, the method comprising administering to said subject a therapeutically effective amount of the antibacterial composition of claim 16.

22. The method of claim 21, wherein said treating comprises reducing one or more bacterial cellular activities selected from the group consisting of: ribosomal peptidyl transferase center (PTC) activity, cellular replication rate, number of replicating cells, protein synthesis rate, and yields of protein synthesis.

23. The method of claim 21, wherein said bacterium is selected from the group consisting of Mycobacterium, and Staphylococcus.

24. (canceled)

25. (canceled)

26. (canceled)

27. (canceled)

28. (canceled)
